(19) United States
(12) Patent Application Publication
(10) Pub. No.: US 2020/0214273 A1
(43) Pub. Date: Jul. 9, 2020

(54) GENOME-EDITED BIRDS

(71) Applicant: THE STATE OF ISRAEL, MINISTRY OF AGRICULTURE & RURAL DEVELOPMENT, AGRICULTURAL RESEARCH ORGANIZATION, Rishon-LeZion (IL)

(72) Inventors: Yuval CINNAMON, Jerusalem (IL); Enbal BEN-TAL COHEN, Rishon-leZion (IL)

(73) Assignee: THE STATE OF ISRAEL, MINISTRY OF AGRICULTURE & RURAL DEVELOPMENT, AGRICULTURAL RESEARCH ORGANIZATION, Rishon-LeZion (IL)

(21) Appl. No.: 16/647,474

(22) PCT Filed: Sep. 17, 2018
(86) PCT No.: PCT/IL2018/051056

§ 371 Date: Mar. 13, 2020

Related U.S. Application Data
(60) Provisional application No. 62/560,218, filed on Sep. 19, 2017.

(51) Int. Cl. A01K 67/027; C12N 15/90; C12N 5/0735

(52) U.S. Cl. CPC A01K 67/0275; C12N 5/0611; C12N 15/907

(57) ABSTRACT

A DNA editing agent is disclosed which comprises a first nucleic acid sequence for eliciting in an inducible manner a lethal phenotype of male chick embryos in an egg of a bird and a second nucleic acid sequence for directing said nucleic acid sequence for effecting said lethal phenotype to a Z chromosome of a cell of the bird. Use of the DNA editing agent is also disclosed.

### FIELD AND BACKGROUND OF THE INVENTION

The present invention, in some embodiments thereof, relates to genome edited birds which lay eggs comprising non-viable male chick embryos.

Sex separation is an important aspect for all avian species production, but in particular for the broiler and essentially all egg layer and turkey production. For broilers and turkeys, sex separation allows a better suited management and feeding according to the needs of both sexes, which are somewhat different than the unisex rearing as done now in most cases. Essentially all commercial hatcheries of pullets that will become table egg laying hens use sex separation of flocks. Male chickens are culled at the hatchery, whereas female chickens are evidently destined for egg production.

Currently, there are three methods available for sexing poultry. Day-old chicks can be sexed either by vent/cloaca sexing, or feather sexing methods. Alternatively, male and female chicks can be reared together until secondary sex characteristics become apparent, then the chicks can be separated based on sex. Vent/cloaca sexing relies on the visual identification of sex, based on the appearance of sex related anatomical structures. Feather sexing is based on feather characteristics that differ between male and female chicks, for example down color pattern, and rapid/slow rate of growth of the wing feathers. The third method relies on the appearance of natural secondary sex characteristics, for example in males the combs and wattles will become larger than those on females.

The vent/cloaca sex determination of day-old chicks is difficult and expensive. Identifying the sex of a bird requires highly skilled personnel. While easier to perform, feather sexing has the disadvantage of being limited to specific genetic crosses of birds. Sexing by secondary sex characteristics is the easiest method to perform but has the disadvantage of requiring birds of both sexes to be reared together for the first weeks after hatch which because of feed costs and feed conversion considerations can be more expensive to the hatchery than the expense of vent/cloaca sexing.

Most importantly, because of the increasing optimization of meat production on one hand and egg production on the other hand, the male pullets of chicken breeds that are optimized for egg productions are no longer suitable or economically attractive for meat production. Therefore, over 500 million male chicks are destroyed every year in the US and Europe only by gassing, electrocution or shredding. This is not only an economic problem, but it has also increasingly become an ethical problem.

Clearly, the commercial hatchery industry has a need for a method that would allow birds to be sorted whilst still in the egg, that does not rely on highly skilled individuals.

Oishi et al., [Scientific Reports 6, Article number: 23980 (2016) doi:10.1038/srep23980] teaches targeted mutagenesis in chicken primordial germ cells using CRISPR/Cas9 system.

Macdonald et al., [PNAS, 2012, p. 1466-1472] teaches genetic modification and germ-line transmission of primordial germ cells using piggyBac and To12 transposons.

US Application No. 20060095980 teaches manipulation of the number of endogenous primordial germ cells of a female bird so as to produce offspring having a higher probability of being males as opposed to females.

Additional background art includes WO 2017094015.

### SUMMARY OF THE INVENTION

According to an aspect of some embodiments of the present invention there is provided a DNA editing agent comprising a first nucleic acid sequence for eliciting in an inducible manner a lethal phenotype of male chick embryos in an egg of a bird and a second nucleic acid sequence for directing the first nucleic acid sequence for effecting the lethal phenotype to a Z chromosome of a cell of the bird.

According to an aspect of some embodiments of the present invention there is provided a cell population comprising cells of a bird which comprise an exogenous polynucleotide which is stably integrated into the Z chromosome of the cells, the exogenous polynucleotide being for eliciting a lethal phenotype in a male offspring of the bird.

According to an aspect of some embodiments of the present invention there is provided a chimeric bird generated according to the method described herein.

According to an aspect of some embodiments of the present invention there is provided a transgenic bird generated using a gamete of the chimeric bird described herein.

According to an aspect of some embodiments of the present invention there is provided a method of reducing the number of male chicks hatching from the fertilized eggs of a bird, wherein an exogenous polynucleotide is stably integrated into the Z chromosome of the bird, the exogenous polynucleotide being for eliciting in an inducible manner a lethal phenotype in a male offspring of the bird, the method comprising exposing the eggs to an inducer which elicits the lethal phenotype, thereby reducing the number of male chicks hatching from the fertilized eggs of a bird.

According to an aspect of some embodiments of the present invention there is provided a chimeric bird comprising the cell population described herein.

According to an aspect of some embodiments of the present invention there is provided a method of generating a chimeric bird comprising administering the cell population described herein to a recipient bird embryo under conditions sufficient to allow at least one of the primordial germ cells (PGCs) of the cell population to colonize a gonad of the recipient bird embryo, thereby generating a chimeric bird.

According to an aspect of some embodiments of the present invention there is provided a DNA editing system comprising:

(i) a first agent comprising a first nucleic acid sequence for eliciting a lethal phenotype in an egg of a bird operatively linked to a recombinase recognition site and a sequence for directing said first nucleic acid sequence for effecting said lethal phenotype to a Z chromosome of a cell of the bird; and

(ii) a second agent comprising a second nucleic acid sequence which encodes a recombinase enzyme and a sequence for directing said second nucleic acid sequence to a Z chromosome of a cell of the bird.

According to an aspect of some embodiments of the present invention there is provided a method of reducing the number of male chicks hatching from the fertilized eggs of a bird comprising:

mating a female bird with a male bird, wherein a first exogenous polynucleotide which is operatively linked to a recombinase recognition site is stably integrated into the Z chromosome of said male bird, said exogenous polynucleotide being for eliciting a lethal phenotype in an egg of a bird and a second exogenous polynucleotide encoding a recombinase enzyme is stably integrated into the Z chromosome of said female bird, or

said first exogenous polynucleotide which is operatively linked to a recombinase recognition site is stably integrated into the Z chromosome of said female bird, said exogenous polynucleotide being for eliciting a lethal phenotype in an egg of a bird and a second exogenous polynucleotide encoding a recombinase enzyme is stably integrated into the Z chromosome of said male bird,

thereby reducing the number of male chicks hatching from the fertilized eggs of a bird.

According to some embodiments of the invention, the eliciting is effected in an inducible manner.

According to some embodiments of the invention, the first nucleic acid sequence encodes a lethality protein which is operatively linked to a nucleotide sequence encoding a switch that controls the expression of the lethality protein, the switch being regulated by an inducer.

According to some embodiments of the invention, the lethality protein is selected from the group consisting of a toxin, a pro-apoptotic protein, a BMP antagonist, an inhibitor of Wnt signaling pathway and an FGF antagonist.

According to some embodiments of the invention, the DNA editing agent is a single molecule.

According to some embodiments of the invention, the first nucleic acid sequence and the second nucleic acid sequence are comprised in non-identical molecules.

According to some embodiments of the invention, the first nucleic acid sequence encodes an endonuclease enzyme that can carry out genome editing which is operatively linked to a nucleotide sequence encoding a switch that controls the expression of the endonuclease protein, the switch being regulated by an inducer.

According to some embodiments of the invention, the second nucleic acid sequence comprises:

(i) a left homology arm (LHA) nucleotide sequence that is substantially homologous to the 5′ region flanking a target gene locus in the Z chromosome of the bird; and

(ii) a right homology arm (RHA) nucleotide sequence that is substantially homologous to the 3′ region flanking the target gene locus in the Z chromosome of the bird.

According to some embodiments of the invention, the inducer is selected from the group consisting of heat, ultrasound, electromagnetic energy and a chemical.

According to some embodiments of the invention, the switch comprises a split recombinase enzyme that combines to form an active enzyme in the presence of the inducer.

According to some embodiments of the invention, the switch comprises an inducible promoter.

According to some embodiments of the invention, the endonuclease enzyme is an RNA-guided DNA endonuclease enzyme.

According to some embodiments of the invention, the DNA editing agent further comprises a nucleotide sequence that encodes for a guide RNA that targets an essential gene of the bird, the nucleotide sequence being operatively linked to a nucleotide sequence encoding the switch.

According to some embodiments of the invention, the essential gene is selected from the group consisting of BMPR1A, BMP2, BMP4 and FGFR1.

According to some embodiments of the invention, the RNA-guided DNA endonuclease enzyme is selected from the group consisting of zinc-finger nucleases (ZFNs), transcription activator-like effector nucleases (TALENs) and caspase 9.

According to some embodiments of the invention, the DNA editing agent further comprises a nucleotide sequence that encodes for a reporter polypeptide.

According to some embodiments of the invention, the inducer is electromagnetic energy.

According to some embodiments of the invention, the electromagnetic energy is a component of visible light.

According to some embodiments of the invention, the component of visible light is blue light.

According to some embodiments of the invention, the bird is selected from the group consisting of chicken, turkey, duck and quail.

According to some embodiments of the invention, the cells comprise primordial germ cells (PGCs).

According to some embodiments of the invention, the cells comprise gametes.

According to some embodiments of the invention, the PGCs are selected from the group consisting of gonadal PGCs, blood PGCs and germinal crescent PGCs.

According to some embodiments of the invention, the method further comprises incubating the chimeric bird to hatch following the administering.

According to some embodiments of the invention, the method further comprises raising the chimeric bird to sexual maturity, wherein the chimeric bird produces gametes derived from the donor PGCs.

According to some embodiments of the invention, the administering is by in-ovo injection.

According to some embodiments of the invention, the cell population are derived from the same avian species as the recipient bird.

According to some embodiments of the invention, the cell population are derived from a different avian species as the recipient bird.

According to some embodiments of the invention, the cell population is administered when the recipient embryo is between about stage IX according to the Eyal-Giladi & Kochav staging system and about stage 30 according to the Hamburger & Hamilton staging system.

According to some embodiments of the invention, the cell population is administered when the recipient embryo is after stage 14 according to the Hamburger & Hamilton staging system.

According to some embodiments of the invention, the first nucleic acid sequence encodes a lethality protein or an endonuclease enzyme that can carry out genome editing.

According to some embodiments of the invention, the sequence for directing said first or said second nucleic acid sequence to the X chromosome comprises:

(i) a left homology arm (LHA) nucleotide sequence that is substantially homologous to the 5′ region flanking a target gene locus in the Z chromosome of the bird; and

(ii) a right homology arm (RHA) nucleotide sequence that is substantially homologous to the 3′ region flanking said target gene locus in the Z chromosome of the bird.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a cartoon illustrating the generation of an optogenetic inducible chicken line from which only female layer chicks will hatch. By crossing wild-type rooster (ZZ) with genetically modified hen (ZW) all the female fertile eggs will carry wild-type ZW chromosomes and the Z chromosome comes from the WT rooster. All the male fertile eggs will carry the ZZ chromosomes in which the red labeled Z is derived from the hen's genome. This is the chromosome to be targeted. Upon blue light illumination of the fertile eggs, the optogenetic system on this chromosome will become active and will activate a death mechanism that will result in early embryonic mortality soon after oviposition. The females which will not be affected by the blue light illumination will grow to adulthood and will lay unfertile eggs for food.

FIG. 2 illustrates a strategy to control gene expression by means of blue light illumination. Two fusion proteins are created Cry2 with the N-terminus of Cre (Cry2-Cre-N-term) and CIBN fused with the C-terminus of Cre (CIBN-Cre-C-term). Without blue light illumination the Cre is inactive. Upon blue light illumination, CRY2 and CIBN form a hetero-dimmer and the two parts of Cre are brought together to form an active Cre enzyme¹¹.

FIG. 3 illustrates the homology arms on chromosome Z. The genomic region downstream to the Hint1 locus (Green arrow) is depicted. The 5′ and 3′ arms, HA-1 and HA-2, respectively are depicted in red. The primers for amplifying the arms are indicated by yellow arrows. In-between the homology arms, on both DNA strands, there are highly unique sequences, specific and adequate for CRISPR-Cas9 (green boxes). Lower part of the figure shows in high detail the region between the two homology arms.

The sequence set forth in SEQ ID NO: 1 is illustrated.

FIGS. 4A-C illustrate targeting vectors according to embodiments of the present invention. The targeting vector contains 3 main elements. The first, the 5′ and 3′ homology arms (HA, light blue) for homologous recombination (HR), flanking the entire insertion cassette, the second, a light-inducible system - in this case the Cry2-CreN and the CIBN-CreC, and the third the lethality gene cassette. (A) The structure of a single targeting vector strategy, containing the 5′ HA, followed by a pGK promoter (Dark blue box) which drive the expression of the Cry2-CreN and the CIBN-CreC genes (red boxes) which are separated by a self-cleaving peptide P2A (purple box). This element is followed by the lethality gene cassette which contains a pGK promoter followed by a LoxP (yellow circle) STOP (red octagon) LoxP sites (LSL), followed by a lethality-inducing gene. This cassette is followed by the 3′ HA (light blue box). Upon light induction, the Cry2-CreN and the CIBN-CreC dimerize to form an active form of Cre. The latter then excises the LSL element, thus allowing expression of the lethality-inducing gene, which leads to embryonic death in all embryos that carry this vector. (B) An alternative approach to A, instead of using the LSL element, a Dio-lox flipping strategy is used (yellow triangles). In between the Dio-Lox sites a GFP followed by polyadenylation site #1 (PA1, gray box) and a lethality gene followed by a different polyadenylation site #2 (PA2) in a reverse orientation, are introduced. In this case, prior to light activation, the pGK promoter drives the expression of the GFP. Upon light activation, the cassette between the Dio-Lox sites flips and the lethality gene is now in the right orientation to be expressed while the GFP is now place in a reverse orientation and it is no longer active. C) An alternative approach to A or B. Following the activation of Cre, the LSL is removed and the Cas9 and sgRNA are expressed. This leads to the introduction of a missense mutation in the coding region of an essential gene, thus inducing embryonic lethality.

FIGS. 5A-F: PGCs line derivation and characterization. A, PGCs culture; B, left, mRNA expression of different pluripotent and germ-cells markers as indicated. Right, representative characterization of sex identification of female PGCs (left, two PCR products of Ribosomal S18 and W chromosome) and male PGCs (right, Ribosomal S18 only). C, PGCs antibody staining of the SSEA1 antigene. D, transfection of PGCs with pCAGG-GFP encoding plasmid using Lipofectamine 2000 reagent. E, PGCs transfection using electroporation the pCAGG-GFP encoding plasmid. F, Gonad (testis) of an embryo, 10 days following transplantation with GFP-expressing cultured PGCs.

FIGS. 6A-C. Designing the sgRNA sites for CRISPR-mediated targeting. Representation of the genomic area on the Z chromosome for the designing for the optimal CRISPR targeting sites. The 12 top sgRNA sequences are presented (Guide #1-#12), of them guides #1 and #3, which partially overlap, in opposite orientations, were chosen. The potential off-targets of guide #1 are presented. Table 1 below provides the sequences of the guide RNAs as presented in FIG. 6B.

TABLE 1

| | SEQ | |
|---|---|---|
| Guide | ID NO: | Sequence |
| 1 | 66 | GCCAAATAAGGCACGTTATC |
| 2 | 67 | AATGTGGAAACGGCCAAATA |
| 3 | 68 | ACCAGATAACGTGCCTTATT |
| 4 | 69 | ACATGACAGCACGATTTTGT |
| 5 | 70 | CTGGTATGAACCAATCAGAG |
| 6 | 71 | TGGTATGAACCAATCAGAGT |
| 7 | 72 | GACCTTGATGCAGAGAAAAC |
| 8 | 73 | CTCCTGTTTTCTCTGCATCA |
| 9 | 74 | GCAGAGAAAACAGGAGAAGA |
| 10 | 75 | AGAAGGATGAGAAAAGAATG |
| 11 | 76 | CTGTCATGTCCCACTCTGAT |
| 12 | 77 | ATGAGAAAAGAATGTGGAAA |

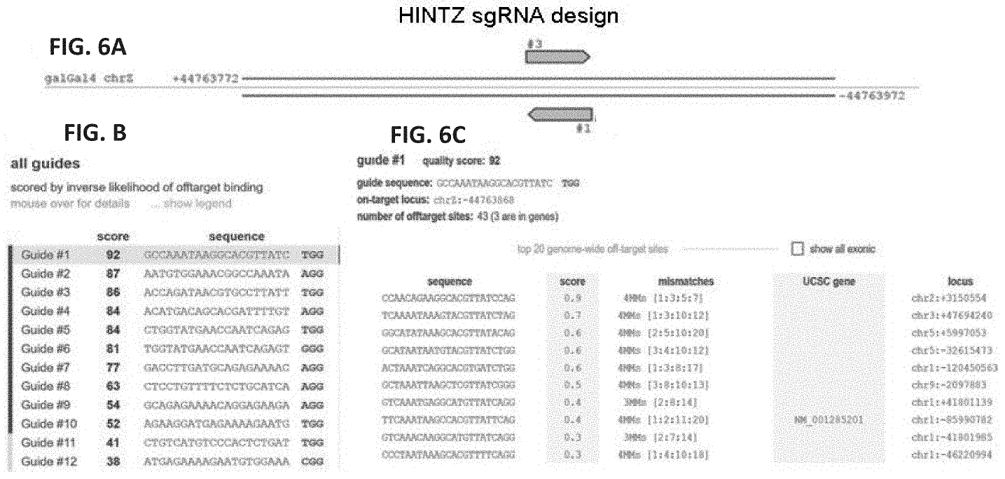

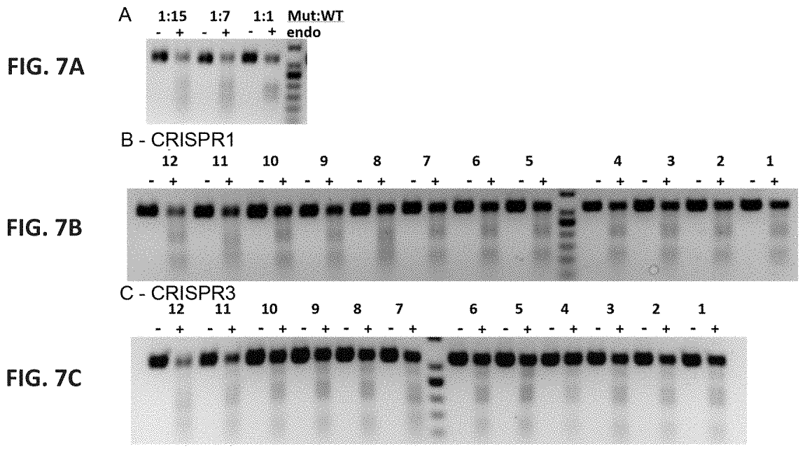

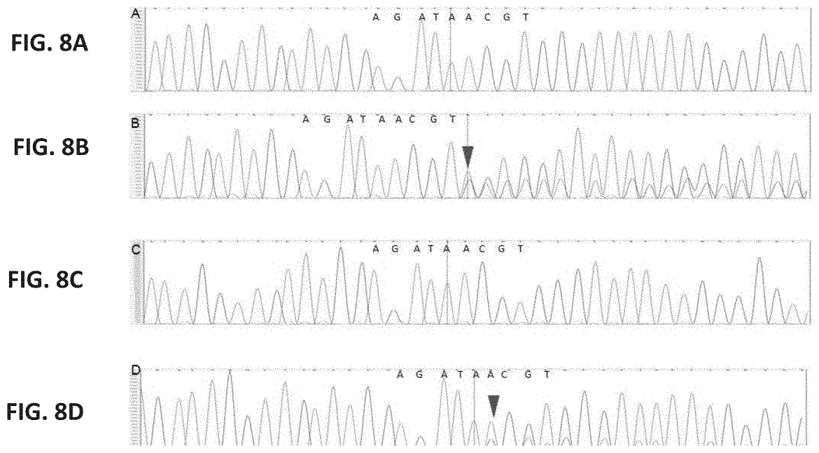

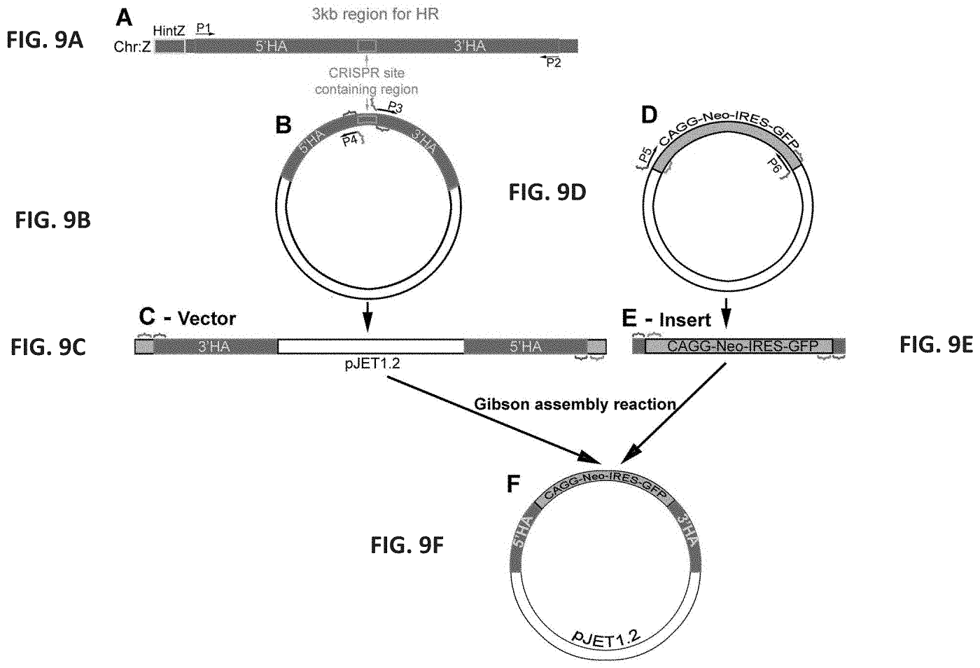

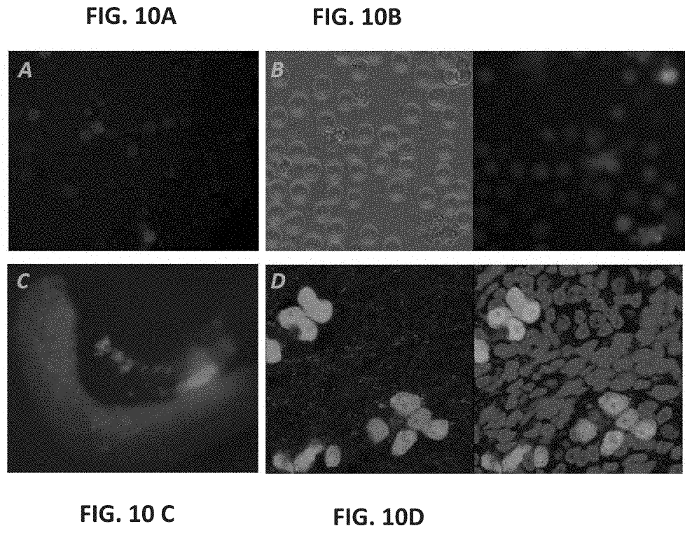

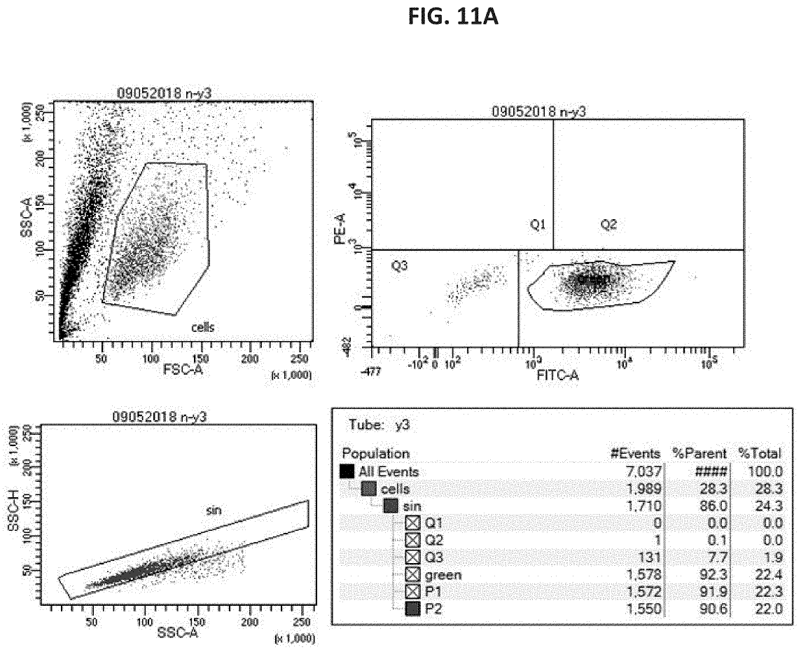

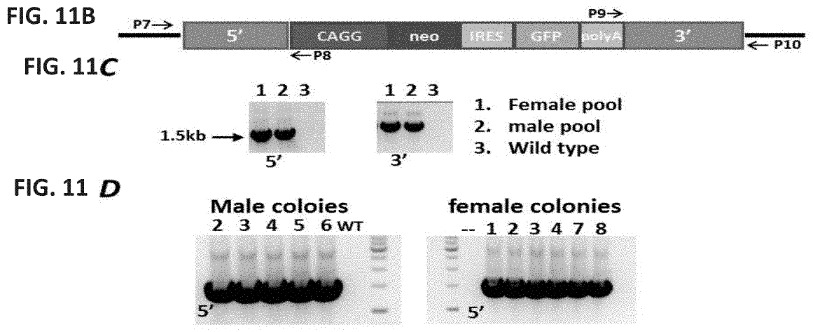

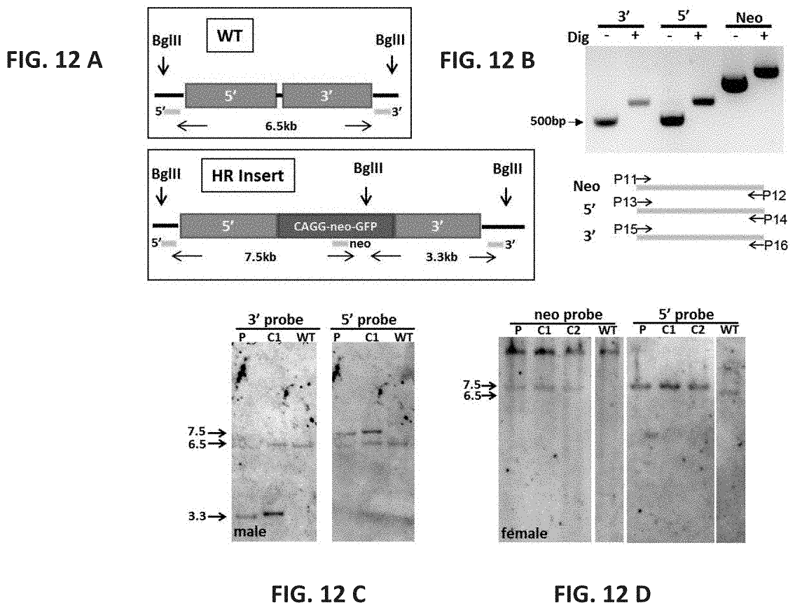

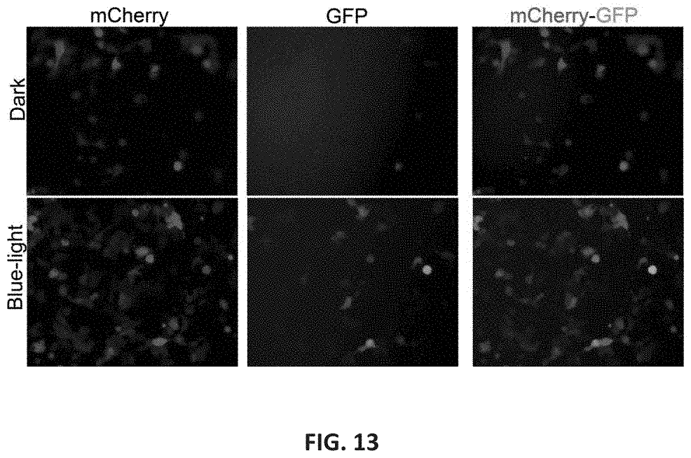

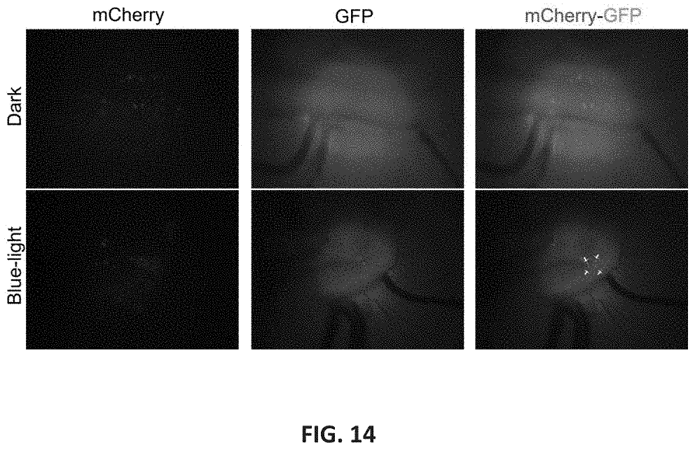

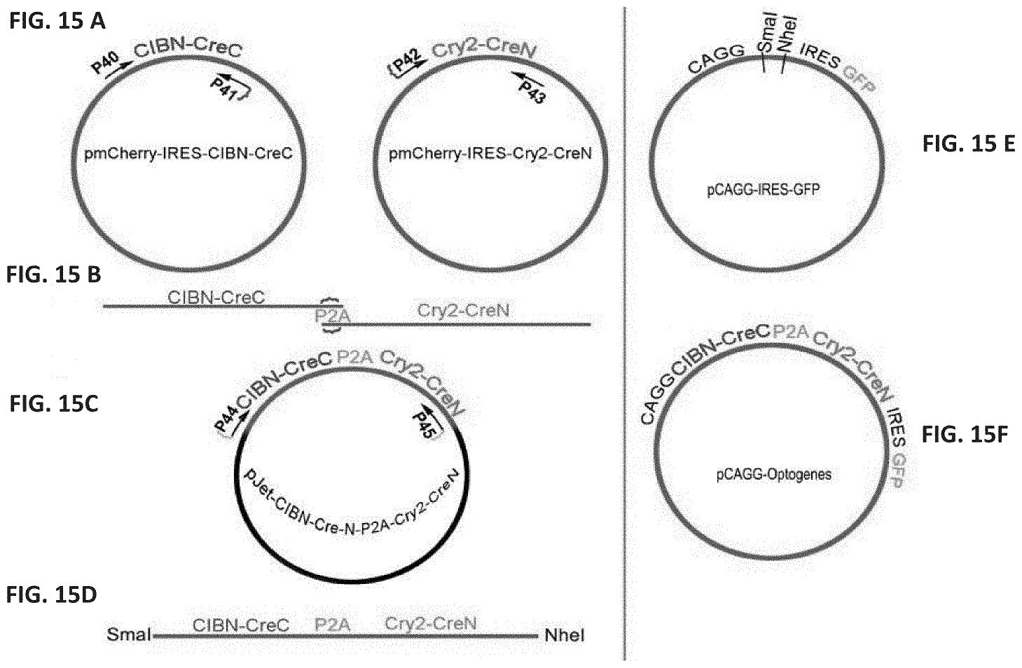

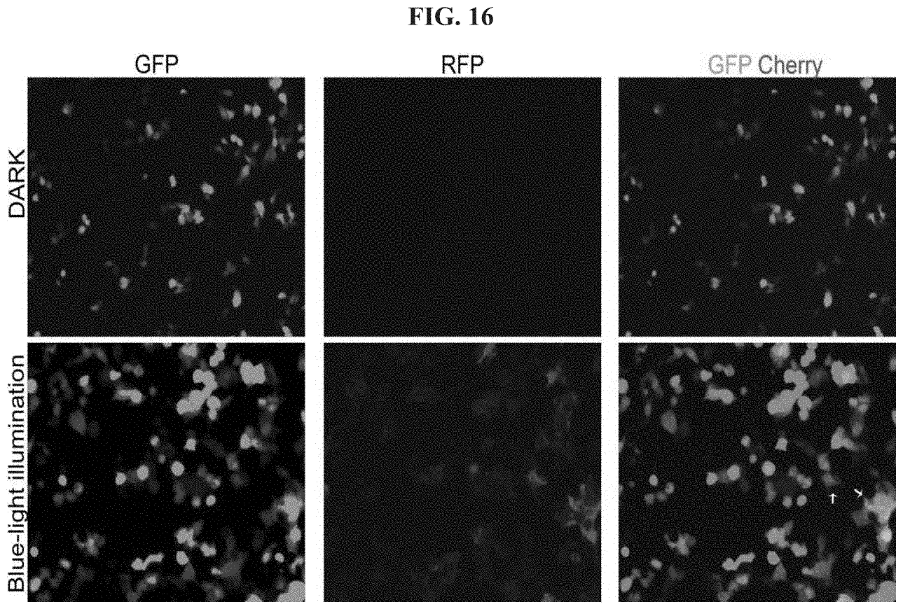

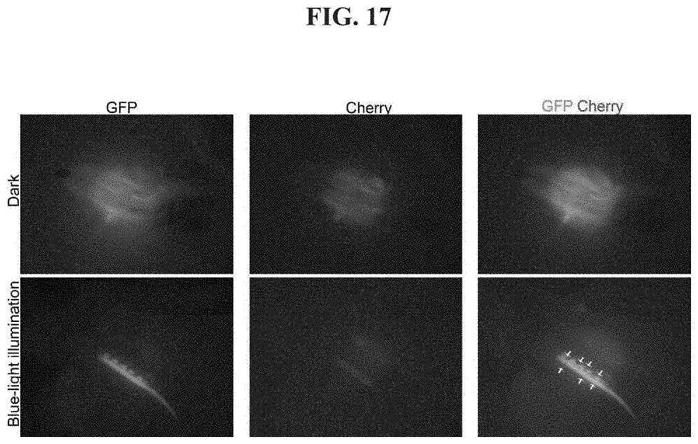

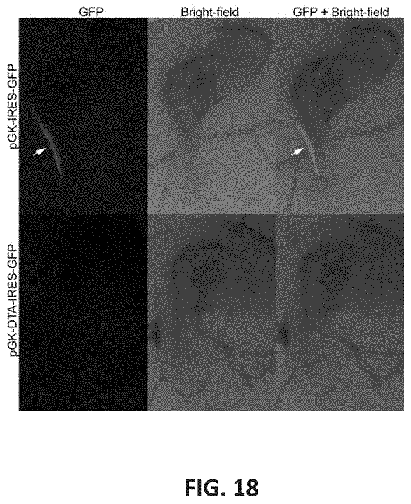

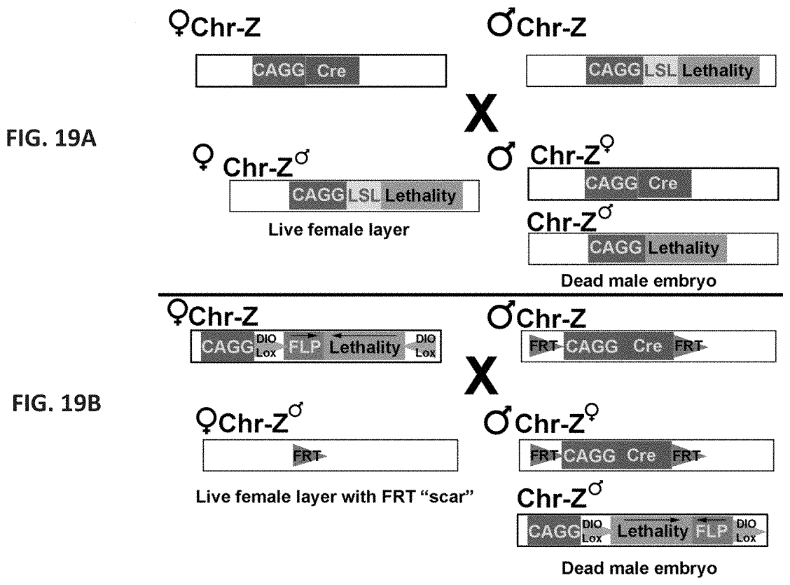

The top 10 results of search for potential off-targets for guide #1 shown in FIG. 6C, the sequences of which are summarized in Table 2.

TABLE 2

| SEQ ID NO: | Sequence |
|---|---|
| 78 | CCAACAGAAGGCACGTTATCCAG |
| 79 | TCAAAATAAAGTACGTTATCTAG |
| 80 | GGCATATAAAGCACGTTATACAG |
| 81 | GCATAATAATGTACGTTATCTGG |
| 82 | ACTAAATCAGGCACGTGATCTGG |
| 83 | GCTAAATTAAGCTCGTTATCGGG |
| 84 | GTCAAATGAGGCATGTTATCAGG |
| 85 | TTCAAATAAGCCACGTTATTCAG |
| 86 | GTCAAACAAGGCATGTTATCAGG |
| 87 | CCCTAATAAAGCACGTTTTCAGG |

FIGS. 7A-C. Validating CRISPR activity using endonuclease assay. A. Positive control of the endonuclease assay using anealed WT 320 bp PCR product and a mutated product at the predicted cleavage site of CRISPR1 at the indicated ratios. B. Endonulease assay on 12 colonies transfected with CRISPR1 plasmid. C. Endonulease assay on 12 colonies transfected with CRISPR3 plasmid. Note that there is a 12 bp distance between the two predicted cleavage sites of CRISPR1 and CRISPR3.

FIGS. 8A-D. Validating CRISPR activity using DNA Sequencing. A. DNA chromatogram of WT genomic region at the predicted cleavage site of CRISPR1, showing a normal sequence as a negative control. B. Sequence of mixture of WT and artificially mutated PCR products showing the appearance of double-peaks (blue arrowhead) after the predicted cleavage site, as a positive control. C. Sequencing of negative colony showing a normal sequence. D. Sequence of positive colony, showing the appearance of double peaks following the CRISPR1 cleavage site (blue arrowhead). The sequence AGATAACGT (SEQ ID NO: 65) is portrayed.

FIGS. 9A-F. Constructing the targeting vector for genome integration in to the Z chromosome. A—Genomic DNA was used as a template for PCR reaction with primers P1 and P2. This region contains 5′HA and 3′HA, flanking the CRISPR-site-containing region. B—The ˜3 kb product, located downstream to the HINTZ locus was ligated to the shuttle vector pJet1.2. This plasmid was used as a template for PCR with primers P3 and P4. These primers have extension sequence (demarcated by color coded curly brackets) which correspond to the equivalent regions on the pCAGG-Neo-IRES-GFP fragment (D). C—The linearized product—the vector, containing the two homology arms, excluding the CRISPR-site-containing region, flanked by sequences which bind the ends of the pCAGG-Neo-IRES-GFP cassette during the Gibson reaction. D—The pCAGG-Neo-IRES-GFP plasmid was used as template for PCR reaction with primers P5 and P6. These primers contain extension sequences (demarcated by color coded curly brackets) which correspond to the equivalent regions on edges of the homology arms. E—The linearized insert cassette flanked by sequences which bind the ends of the homology arms. The Vector and the insert were stitched together to create the final targeting vector plasmid, using the Gibson assembly reaction¹⁰. F—The targeting vector.

FIGS. 10A-D Co-transfection of targeting vector and CRISPR plasmids to PGCs. A. Lipofection-mediated co-transfection to PGCs with CRISPR1 and HR targeting vector plasmids. B. Two weeks after G-418 selection, >99% of the resistant PGCs were positive for GFP. C. Ten days following the injection of targeted PGCs to a host embryo, numerous cells were found to be localized in the gonads (testis). D. The gonads were disected and immuno-stained with anti-GFP antibody, and were scanned using confocal microscope (GFP antibody staining in green and nuclei counterstained with 4′, 6 diamidino-2-phenylindole (DAPI) in blue).

FIGS. 11A-D -PCR verification of HR integration in FACS sorted PGCs. A. FACS sorting of G-418 resistant PGCs. FACS gating was designed to sort singular (sin) GFP-positive cells that were sorted as pool or individual cells in 96 well plate. B. For PCR analysis two sets of primers for the 5′ integration site (P7 and P8) and the 3′ integration site (P9 and P10), were designed. C. Genomic DNA extracted from the pooled cells was used as a template for the PCR and WT DNA served as a negative control. The predicted 1.6 kb and 1.8 kb bands were evident for the correct HR integration in the 5′ and 3′ regions, respectively. D. Genomic DNA extracted from male and female cell colonies, originated from single cell FACS sorted PGCs, was used as a template for the PCR and WT DNA served as a negative control. The predicted 1.6 kb and 1.8 kb bands were evident for the correct HR integration in the 5′ and 3′ regions, respectively.

FIGS. 12A-D—Southern blot analysis of the HR integration. A—schematic representation of the expected BglII cleaved products in Southern blot analysis for the WT allele and the allele which underwent HR integration. The probes used for 5′, 3′ integration sites and for the neo are marked as a yellow squares. The expected product size, following BglII digestion, for each DNA probe are described. B—Preparation of the dig-labeled probes by PCR. dig-labeled probes (+) or un-labeled (−) were analyzed on an agarose gel. Note that dig-labeled products are shifted higher than their actual size, confirming the integration of the dig-labeled nucleotides. The sets of primers used for amplifying the probes are indicated. C—Southern blot analysis with the 5′ and 3′ probes on DNA extracted from pooled and pure colonies of male-derived PGCs. WT DNA extracted from the original line, prior to the HR, served as a negative control. D—Southern blot analysis with the 5′ and the Neo probes on female-derived PGCs. A single 7.5 kb band is evident in both cased, indicating that correct HR occurred and only a single copy of the targeting vector was integrated.

FIG. 13—Validation of the optogenetic system in HEK293 cells, in-vitro. Triple transfection with pmCherry-Cry2-CreN, pmCherry-CIBN-CreC and PB-RAGE-GFP plasmids. Twenty four hours following transfection, experimental group cells were exposed for 15 seconds of blue light illumination while control cells were kept in dark (upper row). Following illumination (lower row), the cells were further incubated for 24 hours. In these cells, GFP expression was evident confirming the activation of the Cre enzyme upon blue-light illumination.

FIG. 14. Validation of the optogenetic system in-ovo in chick embryos, incubated for 54-60 hours prior to electroporation. Triple electroporation to chicken embryos with pmCherry-Cry2-CreN, pmCherry-CIBN-CreC and pB-RAGE-GFP plasmids. Twelve hours following electroporation, experimental group embryos were exposed for 15 sec of blue light illumination in-ovo while control embryos were kept in dark (upper row). Following illumination (lower row), the embryos from both groups were incubated for additional 12 hours. Following incubation, GFP expressing cells were clearly evident in the illuminated group confirming the activation of the optogenetic system and the Cre enzyme upon blue-light illumination in chicken embryos in-ovo.

FIGS. 15A-F—Constructing a single optogene expression vector under the CAGG promoter. The optogenes plasmids pmCherry-CIBN-CreC and pmCherry-Cry2-CreN were used as a template to amplify the optogenes fusion proteins using the P40-P41 and P42-P43 primers, respectively (15A). The two products share overlap sequences at the P2A site which was introduced in primers P41 and P42. This allowed for single-cycle overhang extension PCR, to unite to two fragments (15B), to one, which was ligated to pJet1.2 shuttle vector (15C). Using primers P44 and P45, which contain tails with SmaI and NheI restriction sites, respectively, the product in 15D was generated. This product was digested using the appropriate restriction enzymes and was ligated to pCAGG-IRES-GFP (15E) that was digested with the same enzymes (15F).

FIG. 16. Validating the activity of the pCAGG-Optogene plasmid in HEK293 cells. Co-transfection with pCAGG-Optogene and pB-RAGE-mCherry plasmids. Twenty four hours following transfection, while the negative-control group was kept in the dark (upper row), the experimental group cells were exposed for 15 seconds to blue light illumination (lower row). Following illumination (lower row), the cells were further incubated for 24 hours. In these cells, mCherry expression was evident (white arrows) confirming the activation of the Cre enzyme by the pCAGG-Optogene plasmid, upon blue-light illumination.

FIG. 17. Verification of the single-vector strategy using the pCAGG-Optogenes plasmid in-ovo. Chicken embryos at stage 14-16H&H were co-electroporated with pCAGG-Optogenes and pB-RAGE-mCherry plasmids. The latter plasmid serves as a reporter gene for the activity of the optogenic system. Twelve hours following electroporation, the experimental group embryos (lower row) were exposed for 15 sec to blue light illumination in-ovo while control embryos were kept in the dark (upper row). The embryos were further incubated for 12 hours. Following incubation, GFP expressing cells were clearly evident in both groups, indicating successful electroporation however, only in the illuminated group mCherry-expressing cells were evident, confirming the activation of the optogenetic system and the Cre enzyme upon blue-light illumination in chicken embryos in-ovo.

FIG. 18. Expression of DTA under the pGK promoter inhibits protein synthesis in-ovo. Stage 14-16 H&H embryos were electroporated with either the pGK-IRES-GFP (upper row) or pGK-DTA-IRES-GFP (lower row) expression vector. Negative control embryos widely express GFP (upper row, arrow) indicating normal protein synthesis. DTA expressing cells show no GFP expression (lower row), indicating that protein synthesis in these embryos is inhibited. GFP-only, bright-field-only and GFP overlied on bright-field images are presented.

FIGS. 19A-B illustrate targeting vectors according to additional embodiments of the present invention. In these vectors the activating enzyme (Cre for example) is separated from the lethality gene cassette. In FIG. 19A, the activating enzyme is inserted into the genome of the mother hen and the in-active lethality cassette is inserted on the Z chromosome of the rooster, which is homozygote to this allele. In this case the activation of lethality in male embryos is carried out by crossing the two transgenic parents without the need for light induction. The Cre in all males removes the LSL on the maternal Z chromosome thereby allowing the lethality gene to be expressed, while the female embryo harbors an in-active lethality cassette, thus it is unaffected. Alternatively, the Z chromosome on the mother hen is targeted with Dio-Lox flipping cassette containing the FLP recombinase in the right direction followed by a lethality gene in reverse orientation, driven by the CAGG promoter (FIG. 19B). The rooster, again homozygote to the Z chromosome which is targeted with CAGG-Cre cassette flanked by FRT sites. Upon crossing the two, male embryos will express the Cre located on the paternal Z chromosome, the Dio-Lox cassette flips and the lethality gene becomes active, thereby leading to embryonic lethality of the male embryo. The zygote of the female embryo from this cross contains maternal contribution of the FLP recombinase enzyme that was produced during oogenesis. This maternal protein, removes the CAGG-Cre cassette from the Z chromosome, leaving the female embryo alive with only a FRT “scar” on the Z chromosome.

### DESCRIPTION OF SPECIFIC EMBODIMENTS OF THE INVENTION

The present invention, in some embodiments thereof, relates to genome edited birds which lay eggs comprising non-viable male chick embryos.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Sex separation is an important aspect for all avian species production, but in particular for the broiler and essentially all egg layer and turkey production. For broilers and turkeys, sex separation allows for better suited management and feeding according to the needs of both sexes, which are somewhat different than the unisex rearing as done now in most cases. Essentially all commercial hatcheries of pullets that will become table egg laying hens use sex separation of flocks. Male chickens are culled at the hatchery, whereas female chickens are evidently destined for egg production.

The present inventors have now conceived of a way to eliminate the need for mass culling of billions of male chicks, worldwide. Specifically, the inventors have devised a way to generate a chicken breed in which the “mothers” from the breeding flock will lay fertile eggs, from which only female layers will hatch, while male embryos will cease to develop soon after fertilization. Thus, the need to cull the male chicks will be eliminated and 50% of valuable incubation space will also be saved. Importantly, both the laying hens and the infertile eggs, which are the end-product for the consumer, will be, in every aspect, identical to the layer-hens and food-eggs which are currently produced by the industry (i.e. non-genetically modified).

Whilst reducing the present invention to practice, the present inventors designed a targeting vector, containing the HAs flanking the Neo and GFP genes under the CAGG promoter and showed that it undergoes correct HR to the Z chromosome as was validated using both PCR (FIGS. 11A-D) and Southern blot (FIGS. 12A-D). Moreover, the single-vector strategy of the optogenic system that was constructed on the pCAGG-Optogene vector was found to be active both in-vitro and in-ovo, in living chick embryo (FIG. 14). Lastly, expression of DTA in chicken embryos resulted in inhibition of protein synthesis (FIG. 18).

Thus, according to a first aspect of the present invention, there is provided a DNA editing agent comprising a first nucleic acid sequence for eliciting in an inducible manner a lethal phenotype of male chick embryos in an egg of a bird and a second nucleic acid sequence for directing the first nucleic acid sequence for effecting the lethal phenotype to a Z chromosome of a cell of the bird.

As used herein, the terms “bird” and “avian species” refer to any avian species, including but not limited to chicken, turkey, duck, geese, quail, pheasant, and ostrich.

As used herein, the term “egg” refers to an avian egg that contains a live embryonic bird. Thus, the term “egg” is intended to refer to a fertilized avian egg, in one embodiment an egg containing an avian embryo that is capable of undergoing normal embryogenesis.

The DNA editing agent (comprised in a single nucleic acid construct or a combination of nucleic acid constructs) comprises targeting sequences which brings about stable integration of the first nucleic acid sequence into the Z chromosome of a cell of the bird. The DNA editing agent may be constructed using recombinant DNA technology well known to persons skilled in the art.

The targeting sequences are selected such that the first nucleic acid sequence integrates specifically into the Z chromosome and not any other chromosome of the cell. Furthermore, the targeting sequence is selected depending on what method is being relied upon to integrate the first nucleic acid sequence into the chromosome. Methods of integrating nucleic acid sequences into the chromosome are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases and genome editing by engineered nucleases. PB transposases are also contemplated. Agents for introducing nucleic acid alterations to a gene of interest can be designed publically available sources or obtained commercially from Transposagen, Addgene and Sangamo Biosciences.

In one embodiment, the DNA editing agent relies on spontaneous homologous recombination to insert the first nucleic acid sequence into the Z chromosome of the cell. In this embodiment, the DNA editing agent comprises homology arms that serve as targeting sequences.

The DNA editing agent may be flanked with two arms that are homologous or show homology or identity of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to at least one nucleic acid sequence comprised within the target loci within the Z chromosome, that serves as the integration site to facilitate specific integration via HDR.

The homology arms correspond to a genomic sequence which is present on the Z chromosome. Preferably, the genomic sequence is downstream of a gene which is transcriptionally active (for example downstream to the Hint1 gene). The homology arms are typically at least 500 nucleotides long, for example between 500-3000 nucleotides long. Typically, the required size of the homology arms relies on the length of the cassettes which are flanked by these arms. Smaller cassettes require shorter arms and vice versa. Homologous recombination can occur spontaneously. Another contemplated target is Is11 (Gene ID 369383), also on chromosome Z which is expressed starting from early stages of embryogenesis.

Following is a description of various additional exemplary methods which may be used by the DNA editing agent to integrate the first nucleic acid sequence into the Z chromosome according to specific embodiments of the present invention and a description of the targeting sequences that are required to carry out this purpose.

Genome Editing using engineered endonucleases—this approach refers to a reverse genetics method using artificially engineered nucleases to cut and create specific double-stranded breaks at a desired location(s) in the genome (i.e. on the Z chromosome), which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, MT et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally, Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in indels which are small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas system—Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the CRISPR design tool from Feng Zhang lab's, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are publicly available such as the px330 plasmid from Addgene. Additionally, mRNA encoding Cas9 and the gRNA can be inserted to the target cells as well as recombinant Cas9 protein in complex with the gRNA (i.e. insert the RNP complex into the cell).

Genome editing using recombinant adeno-associated virus (rAA V) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and Western blot analysis and immunohistochemistry.

The DNA editing agent may encode a reporter protein that is readily detectable either by its presence or activity, including, but not limited to, luciferase, fluorescent protein (e.g., green fluorescent protein), chloramphenicol acetyl transferase, beta-galactosidase, secreted placental alkaline phosphatase, beta-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and typically without the need to kill the cells for signal analysis. In certain instances, a reporter gene encodes an enzyme, which produces a change in fluorometric properties of the host cell, which is detectable by qualitative, quantitative, or semi-quantitative function or transcriptional activation. Exemplary enzymes include esterases, β-lactamase, phosphatases, peroxidases, proteases (tissue plasminogen activator or urokinase) and other enzymes whose function can be detected by appropriate chromogenic or fluorogenic substrates known to those skilled in the art or developed in the future.

In addition, one ordinarily skilled in the art can readily design a DNA editing agent which includes a positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT), Diphtheria toxin (DT) and adenine phosphoribosyltransferase (ARPT).

In order to facilitate homologous recombination, a chemical inhibitor of NHEJ such as SCR7 pyrazine may be used to enhance CRISPR genome editing-mediated HR efficiency. As mentioned, the DNA editing agent of this aspect of the present invention comprises a first nucleic acid sequence for eliciting in an inducible manner a lethal phenotype of male chicks hatching from an egg of a bird.

Preferably, the male embryos do not survive in the egg past early blastulation stages known as stages X-XIII EG&K (Eyal-Giladi and Kochav, 1976).

In one embodiment, the first nucleic acid sequence may encode a lethality protein which is operatively linked to a nucleotide sequence encoding a switch that controls the expression of the lethality protein, wherein the switch is regulated by an inducer.

As used herein, the term “lethality protein” refers to a protein that is lethal to an avian embryo (e.g. male embryo) thus preventing the hatching of a live male bird from the egg. Examples of lethality proteins include, but are not limited to a toxin, a cytotoxic protein, pro-apoptotic protein, a BMP antagonist, an inhibitor of Wnt signaling and an FGF antagonist.

Exemplary toxins contemplated by the present invention include, but are not limited to Pseudomonas exotoxin (GenBank Accession No. ABU63124), diphtheria toxin (GenBank Accession No. AAV70486) and ricin toxin (GenBank Accession No. EEF27734).

Exemplary cytotoxic proteins include, but are not limited to interleukin 2 (GenBank

Accession No. CAA00227), CD3 (GenBank Accession No. P07766), CD16 (GenBank Accession No. NP_000560.5), interleukin 4 (GenBank Accession No. NP_000580.1) and interleukin 10 (GenBank Accession No. P22301).

Exemplary pro-apoptotic proteins contemplated by the present invention include but are not limited to Drosophila Reaper & Grim, known to induce apoptosis also in mammalian cells [McCarthy, J. V & Dixit, V. M. Apoptosis induced by Drosophila reaper and grim in a human system. Attenuation by inhibitor of apoptosis proteins (cIAPs). J. Biol. Chem. 273, 24009-15 (1998)] and those proteins that activate apoptosis such as CASP3 [Julien, 0. & Wells, J. A. Caspases and their substrates. Cell Death Differ. (2017). doi:10.1038/cdd.2017.44].

Other contemplated lethality proteins are those which interfere with basic stages of early embryogenesis, such as N-cadherin which is expressed at early stages of gastrulation. Expression of dominant-negative form of this adhesion molecule will result in early embryonic mortality.

Additional lethality proteins are those which interfere with essential signaling pathways such as the BMP & FGF pathways. Over-expression of the BMP4 antagonist for example—Noggin, will stop the embryogenic process and will result in early embryonic mortality.

In another embodiment, the first nucleic acid sequence may encode an endonuclease enzyme that can carry out genome editing which is operatively linked to a nucleotide sequence encoding a switch that controls the expression of the endonuclease protein, the switch being regulated by an inducer.

Examples of endonuclease proteins include Zinc finger nucleases (ZFNs), transcription-activator like effector nucleases (TALENs) and CRISPR/Cas system, each of which being further described herein above.

In this embodiment, the nucleic acid sequence further comprises a targeting sequence (or guide sequence) which targets the endonuclease to disrupt a gene which is essential for survival of the embryo. In this way, a lethal phenotype is brought about in the embryo and a live (male) chick cannot hatch from the egg.

Examples of essential genes which may be disrupted include, but are not limited to BMPR1A (Gene ID: 396308), BMP2 (Gene ID: 378779), BMP4 (Gene ID: 396165) and FGFR1 (Gene ID: 396516).

The DNA editing agent of this aspect of the present invention will typically comprise a promoter which is operatively linked to drive the expression of the lethality protein or the endonuclease enzyme.

Examples of promoters that may be used in the DNA editing agent include but are not limited to PGK promoter of lentiviruses, CMV promoter, human Synapsin I promoter (hSyn), and the CAG promoter as in the pCAGGS expression vector.

The promoters and other sequences which control expression of the lethality protein or endonuclease enzyme are selected such that there is sufficient expression of the protein so as to bring about a lethality phenotype in male chick embryos.

Irrespective of whether the DNA editing agent encodes an endonuclease or a lethality protein, expression of these effector proteins are regulated by a switch which is comprised (e.g. encoded) in the DNA editing agent.

As used herein, the term “switch” refers to a single component or a set of components that act in a coordinated manner to affect a change, encompassing all aspects of biological function such as activation, repression, enhancement or termination of that function. In one embodiment, switches relate to inducible and repressible systems used in gene regulation. In general, an inducible system may be off unless there is the presence of some molecule or energy form (called an inducer) that allows for gene expression. The molecule is said to “induce expression”. The manner by which this happens is dependent on the control mechanisms as well as differences in cell type. A repressible system is on except in the presence of some molecule or energy form (called a corepressor) that suppresses gene expression. The molecule is said to “repress expression”. The manner by which this happens is dependent on the control mechanisms as well as differences in cell type.

The term “inducible” as used herein may encompass all aspects of a switch irrespective of the molecular mechanism involved. Accordingly, a switch as comprehended by the invention may include but is not limited to antibiotic based inducible systems, electromagnetic energy based inducible systems, small molecule based inducible systems, nuclear receptor based inducible systems and hormone based inducible systems. In some embodiments the switch may be a tetracycline (Tet)/DOX inducible system, a light inducible systems, a Abscisic acid (ABA) inducible system, a cumate repressor/operator system, a 40HT/estrogen inducible system, an ecdysone-based inducible systems or a FKBP12/FRAP (FKBP12-rapamycin complex) inducible system.

It will be appreciated that the inducer should be able to penetrate the egg of the bird. Furthermore, the inducer itself should not be toxic to or alter the development of the embryo inside the egg.

Exemplary inducers contemplated by the present invention include, but are not limited to heat, ultrasound, electromagnetic energy and a chemical. The inducers may be delivered to the egg during the process of egg production inside the hen's body prior to oviposition.

According to a specific embodiment, the switch is induced using electromagnetic energy (for example a component of visible light). The component of visible light may have a wavelength in the range of 450 nm-700 nm or between 450 nm-500 nm, i.e. blue light. The blue light may be of intensity of at least 0.2 mW/cm², or of at least 4 mW/cm².

The component of visible light may have a wavelength in the range of 620 nm-700 nm, i.e. red light.

Single or multiple applications of visible light, in any order and in any combination are contemplated. The visible light may be delivered as single or multiple continuous applications, or as pulses (pulsatile delivery).

Examples of such optogenetic switches are described in Muller et al., Biol Chem. 2015 February; 396(2):145-52. doi: 10.1515/hsz-2014-0199; Motta Mena et al., Nat Chem Biol. 2014 March; 10(3): 196-202; and WO 2014018423, the contents of each being incorporated herein by reference.

In one embodiment, the switch drives the expression of an effector molecule which is expressed when the switch is turned on using the inducer. Exemplary promoters for driving the expression of the effector molecule include but are not limited to PGK promoter of lentiviruses, the pCAGG promoter, CMV promoter, EF1-a promoter, and the human Synapsin I promoter (hSyn).

According to a particular embodiment the effector molecule is a site specific recombinase.

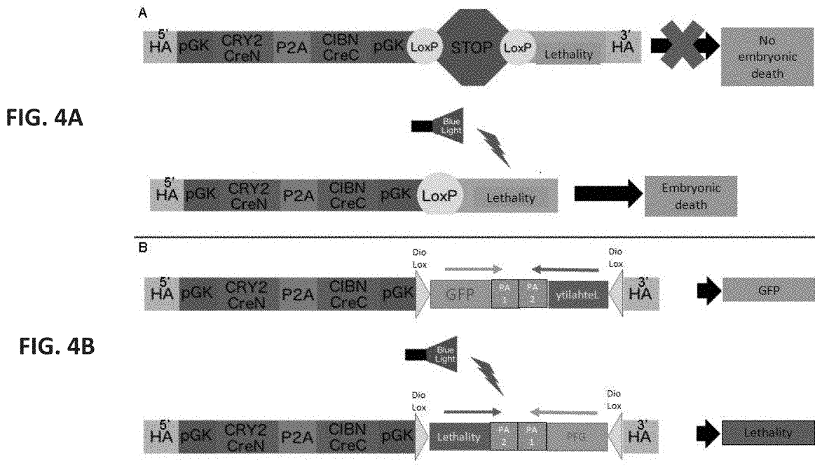

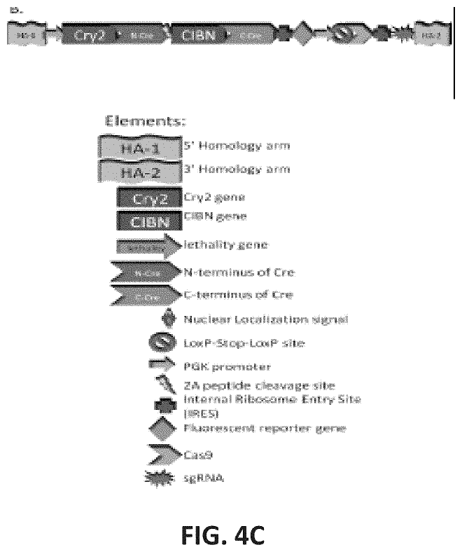

Exemplary optogenetic switches are illustrated in FIGS. 4A-C, each of which utilize the light-sensitive dimerizing protein domains cryptochrome 2 (CRY2) and CIB1 from Arabidopsis thaliana and a site specific recombinase as the effector molecule. The CRY2 is fused in frame to one half of a Cre recombinase whereas the CIB1 is fused in frame to the other half of a Cre recombinase—i.e. a split recombinase enzyme. Thus, when the inducer is provided (blue light is shone), the CRY2 and the CIB1 heterodimerize producing a functional Cre recombinase which is able to carry out site specific recombination.

Site specific recombination is further described herein below.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively.

Additionally, contemplated recombinase recognition sites include, but are not limited to Lox511, Lox5171, Lox2272, m2, Lox71, Lox66, FRT, F₁, F₂, F₃, F₄, F₅, FRT(LE), FRT(RE), attB, attP, attL, and attR.

For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences. An exemplary targeting vector using the Cre/Lox recombination is illustrated in FIGS. 4A and C. An exemplary targeting vector using the Flp/FRT recombination is illustrated in FIGS. 4B.

Other methods of energy activation are contemplated, in particular, electric field energy and/or ultrasound which have a similar effect. If necessary, the proteins pairings of the switch may be altered and/or modified for maximal effect by another energy source.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 and 500 milliseconds, preferably between 1 and 100 milliseconds. The electric field may be applied continuously or in a pulsed manner for 5 about minutes. As used herein, ‘electric field energy’ is the electrical energy to which a cell is exposed. Preferably the electric field has a strength of from about 1 Volt/cm to about 10 kVolts/cm or more under in vivo conditions (see W097/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Notably, an alternative approach can be used, in which the activating enzyme (i.e. recombinase enzyme such as Cre for example) is separated from the lethality gene cassette. In this case, the activating enzyme is inserted into the genome of either the male or female bird and the in-active lethality cassette is inserted on the Z chromosome of the corresponding sex of the bird. In this case, the activation of lethality in male embryos is performed merely by crossing the two transgenic parents.

Thus, according to another aspect of the present invention there is provided a DNA editing system comprising:

(i) a first agent comprising a first nucleic acid sequence for eliciting a lethal phenotype in an egg of a bird operatively linked to a recombinase recognition site and a sequence for directing said first nucleic acid sequence for effecting said lethal phenotype to a Z chromosome of a cell of the bird; and

(ii) a second agent comprising a second nucleic acid sequence which encodes a recombinase enzyme and a sequence for directing said second nucleic acid sequence to a Z chromosome of a cell of the bird.

The DNA editing agents and systems of the present invention may also comprise a reporter gene, which encodes a reporter polypeptide that is readily detectable either by its presence or activity, including, but not limited to, luciferase, fluorescent protein (e.g., green fluorescent protein), chloramphenicol acetyl transferase, beta-galactosidase, secreted placental alkaline phosphatase, beta-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and typically without the need to kill the cells for signal analysis. In certain instances, a reporter gene encodes an enzyme, which produces a change in fluorometric properties of the host cell, which is detectable by qualitative, quantitative, or semiquantitative function or transcriptional activation. Exemplary enzymes include esterases, beta-lactamase, phosphatases, peroxidases, proteases (tissue plasminogen activator or urokinase) and other enzymes whose function can be detected by appropriate chromogenic or fluorogenic substrates known to those skilled in the art or developed in the future. The reporter gene may report on successful integration of the construct into the Z chromosome.

Other components of the DNA editing agent include self-cleaving peptides such as the 2A, including but not limited to P2A, T2A, E2A (Wang et al., Scientific Report 5, Article 16273 (2015), or internal ribosome entry site (IRES) sequences.

The DNA editing agent may be constructed in a viral vector (using a single vector or multiple vectors). Such vectors are commonly used in gene transfer and gene therapy applications. Different viral vector systems have their own unique advantages and disadvantages. Viral vectors that may be used to integrate the first nucleic acid sequence of the present invention into the Z chromosome include but are not limited to adenoviral vectors, adeno-associated viral vectors, alphavirus vectors, herpes simplex viral vectors, and retroviral vectors, described in more detail below. According to a particular embodiment, the vector is a lentiviral vector.

A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Preferably, the codons encoding the proteins of the DNA editing agent are “optimized” codons, i.e., the codons are those that appear frequently in, e.g., highly expressed genes in the bird species, instead of those codons that are frequently used by, for example, an influenza virus. Such codon usage provides for efficient expression of the protein in avian cells. Codon usage patterns are known in the literature for highly expressed genes of many species (e.g., Nakamura et al, 1996; Wang et al, 1998; McEwan et al. 1998).

As mentioned, the DNA editing agent (or system) is used for the generation of female chickens that are only capable of producing viable female offspring and not viable male offspring.

As a first step, the DNA editing agent is introduced into either primordial germ cells of the bird or directly into sperm cells of the bird.

Approaches for introducing nucleic acids of interest into recipient cells are known and include lipofection, transfection, microinjection, electroporation, transformation and microprojectic techniques, etc.

Thus, according to another aspect of the present invention there is provided a cell population comprising cells (e.g. primordial germ cells or gametes) of a bird which comprise an exogenous polynucleotide which is stably integrated into the Z chromosome of the cells, said exogenous polynucleotide being for eliciting (e.g. in an inducible manner) a lethal phenotype in a male offspring of the bird (and not in the female offspring of the bird).

As used herein, the terms “primordial germ cell” and “PGC” refer to a diploid cell that is present in the early embryo and that can differentiate/develop into haploid gametes (i.e. spermatozoa and ova) in an adult bird. Primordial germ cells can be isolated from different developmental stages and from various sites in a developing avian embryo as is known to those of skill in the art including, but not limited to the genital ridge, the developing gonad, the blood, and the germinal crescent. See e.g. Chang et al., Cell Biol Int 21:495-9, 1997; Chang et al., Cell Biol Int 19:143-9, 1995; Allioli et al., Dev Biol 165:30-7, 1994; Swift, Am J Physiol 15:483-516; and PCT International Publication No. WO 99/06533. The genital ridge is a section of a developing embryo that is known to a person of ordinary skill in the art. See e.g., Strelchenko, Theriogenology 45: 130-141, 1996; Lavoir, J Reprod Dev 37: 413-424, 1994. Typically, PGCs are stain positively in the periodic acid-Schiff (PAS) technique. In several species, PGCs can be identified using an anti-SSEA antibody (one notable exception being turkeys, the PGCs from which do not display the SSEA antigen). Various techniques for isolation and purification of PGCs are known in the art, including the concentration of PGCs from blood using Ficoll density gradient centrifugation (Yasuda et al., J Reprod Fertil 96:521-528, 1992).

The in vitro culture of PGCs is possible using a medium containing chicken and bovine serum, conditioned media, feeder cells and growth factors such as FGF2 (van de Lavoir et al. 2006, Nature 441:766-769. doi:10.1038/nature04831); Choi et al. 2010, PLoS ONE 5:e12968. doi:10.1371/journal.pone.0012968; MacDonald et al.2010. PLoS ONE 5:e15518. doi:10.1371/journal.pone.0015518). Recently, it has been shown that a feeder replacement medium containing growth factors to activate the FGF, insulin and TGF-β signaling pathways could be used to propagate PGCs (Whyte et al. 2015, Stem Cell Rep 5:1171-1182.

doi:10.1016/j.stemcr.2015.10.008). Furthermore in this report, use of ovotransferrin as a replacement for the iron-carrying proteins present in avian serum permitted feeder-free and serum-free propagation of PGCs, with cells maintaining a high rate of proliferation.

Primordial germ cells (PGCs) can be provided and formulated for carrying out the presently disclosed subject matter by any suitable technique, and stored, frozen, cultured, or the like prior to use as desired. For example, primordial germ cells can be collected from donor embryos at an appropriate embryonic stage. Stages of avian development are referred to herein by one of two art-recognized staging systems: the Eyal-Giladi & Kochav system (EG&K; see Eyal-Giladi & Kochav, Dev Biol 49:321-327, 1976), which uses Roman numerals to refer to pre-primitive streak stages of development, and the Hamburger & Hamilton staging system (H&H; see e.g., Hamburger & Hamilton, J Morphol 88:49-92, 1951), which uses Arabic numerals to reference to post-laying stages. Unless otherwise indicated, the stages referred to herein are stages as per the H&H staging system.

For example, PGCs can be isolated at stage 4, or the germinal crescent stage, through stage 30, with cells being collected from blood, genital ridge, or gonad in the later stages. The primordial germ cells are, in general, twice the size of somatic cells and easily distinguished and separated therefrom on the basis of size. Male (or homogametic) primordial germ cells (ZZ) can be distinguished from heterogametic primordial germ cells (Zw) by any suitable technique, such as collecting germ cells from a particular donor and typing other cells from that donor, the collected cells being of the same chromosome type as the typed cells.

An alternative to the use of PGCs is the direct transfection of spermatozoa using a DNA editing agent disclosed herein—see for example Cooper et al., 2016 Transgenic Res 26:331-347, doi:10.1007/s11248-016-0003-0.

To produce chimeric birds from PGCs edited in vitro, the exogenous edited cells are injected intravenously into surrogate host embryos, at a stage when their endogenous PGCs are migrating to the genital ridge. The “donor” PGCs may be of the same species as the surrogate host embryo or of a different species. The edited “donor” PGCs must remain viable and in one embodiment, out-compete the endogenous PGCs if they are to colonise the forming gonad and transmit the edited chromosome(s) through the germline. To provide donor PGCs with an advantage, the number of endogenous PGCs can be reduced by chemical or genetic ablation (Smith et al. 2015, Andrology 3:1035-1049. doi:10.1111/andr.12107). Exposing the blastoderm of surrogate embryos to emulsified Busulfan has been shown to increase germline transmission of donor PGCs to over 90%, though this rate drops significantly if PGCs have been cultured or cryopreserved (Nakamura et al. 2008, Reprod Fertil Dev 20:900-907. doi:10.1071/ RD08138; Naito et al. 2015, Anim Reprod Sci. 153:50-61. doi:10.1016/j.anireprosci.2014.12.003). Other methods of skewing the ratio of edited PGCs to native PGCs are described in US Application No. 20060095980.

Optionally, the PGCs may be transplanted into adult gonads as known in the art, see for example Trefil et al., 2017 Sci Rep, Oct 27;7(1):14246 doi: 10.1038/s41598-017-14475-w.

The genetically modified cells (e.g. PGCs) can be formulated for administration to other birds by dissociating the cells (e.g., by mechanical dissociation) and intimately admixing the cells with a pharmaceutically acceptable carrier (e.g., phosphate buffered saline solution). The primordial germ cells are in one embodiment gonadal primordial germ cells, and in another embodiment blood primordial germ cells (“gonad” or “blood” referring to their tissue of origin in the original embryonic donor). The primordial germ cells administered can be heterogametic (Zw) or homogametic (ZZ). PGCs can be administered in physiologically acceptable carrier, in one embodiment at a pH of from about 6 to about 8 or 8.5, in a suitable amount to achieve the desired effect (e.g., 100 to 30,000 PGCs per embryo). The PGCs can be administered free of other ingredients or cells, or other cells and ingredients can be administered along with the PGCs.

Administration of the primordial germ cells to the recipient animal in-ovo can be carried out at any suitable time at which the PGCs can still migrate to the developing gonads. In one embodiment, administration is carried out from about stage IX according to the Eyal-Giladi & Kochav (EG&K) staging system to about stage 30 according to the Hamburger & Hamilton staging system of embryonic development, and in another embodiment, at stage 15. For chickens, the time of administration is thus during days 1, 2, 3, or 4 of embryonic development: in one embodiment day 2 to day 2.5. Administration is typically by injection into any suitable target site, such as the region defined by the amnion (including the embryo), the yolk sac, etc. In one embodiment, injection is into the embryo itself (including the embryo body wall), and in alternative embodiments, intravascular or intracoelomic injection into the embryo can be employed. In other embodiments, the injection is performed into the heart. The methods of the presently disclosed subject matter can be carried out with prior sterilization of the recipient bird in ovo (e.g. by chemical treatment using Busulfan of by gamma or X-ray irradiation). As used herein, the term “sterilization” refers to render partially or completely incapable of producing gametes derived from endogenous PGCs). When donor gametes are collected from such a recipient, they can be collected as a mixture with gametes of the donor and the recipient. This mixture can be used directly, or the mixture can be further processed to enrich the proportion of donor gametes therein.

The in-ovo administration of the primordial germ cells can be carried out by any suitable technique, either manually or in an automated manner. In one embodiment, in-ovo administration is performed by injection. The mechanism of in- ovo administration is not critical, but it is the mechanism should not unduly damage the tissues and organs of the embryo or the extraembryonic membranes surrounding it so that the treatment will not unduly decrease hatch rate. A hypodermic syringe fitted with a needle of about 18 to 26 gauge is suitable for the purpose. A sharpened pulled glass pipette with an opening of about 20-50 microns diameter may be used. Depending on the precise stage of development and position of the embryo, a one-inch needle will terminate either in the fluid above the chick or in the chick itself. A pilot hole can be punched or drilled through the shell prior to insertion of the needle to prevent damaging or dulling of the needle. If desired, the egg can be sealed with a substantially bacteria-impermeable sealing material such as wax or the like to prevent subsequent entry of undesirable bacteria. It is envisioned that a high-speed injection system for avian embryos will be particularly suitable for practicing the presently disclosed subject matter. All such devices, as adapted for practicing the methods disclosed herein, comprise an injector containing a formulation of the primordial germ cells as described herein, with the injector positioned to inject an egg carried by the apparatus in the appropriate location within the egg. In addition, a sealing apparatus operatively connected to the injection apparatus can be provided for sealing the hole in the egg after injection thereof. In another embodiment, a pulled glass micropipette can be used to introduce the PGSc into the appropriate location within the egg—for example directly into the blood stream, either to a vein or an artery or directly into the heart.

Once the eggs have been injected with the modified PGCs, the chimeric embryo is incubated to hatch. It is raised to sexual maturity, wherein the chimeric bird produces gametes derived from the donor PGCs.

The gametes, (either eggs or sperm) from the chimeras (or from material that has been directly genetically manipulated, as described herein above) are then used to raise founder chickens (F1). Molecular biology techniques known in the art (e.g. PCR and/or Southern blot) may be used to confirm germ-line transmission. F1 chickens may be back-crossed to generate homozygous ZZ carrier males and carrier females (F2).

Gametes from founder chickens—F2, can then be used to expand the breeding colonies. The colonies are typically grown until sexual maturity. Fertile eggs obtained from these flocks may be tested for early embryonic mortality of the males by exposure to an inducer which elicits the lethal phenotype, (e.g. blue light illumination). Following induction, the eggs are incubated (for example for 8 days) and screened (e.g. by light-candling) to detect for early embryonic mortality.

It is expected that during the life of a patent maturing from this application many relevant DNA editing tools will be developed and the scope of the term DNA editing agent is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### The Workflow

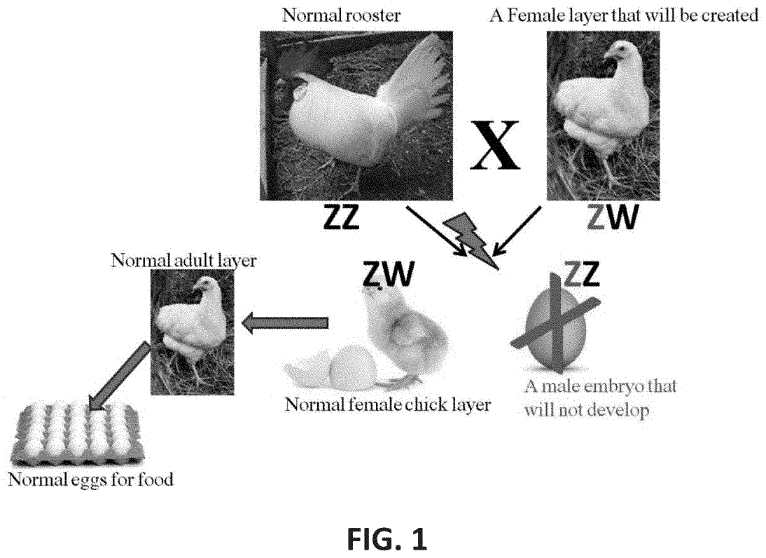

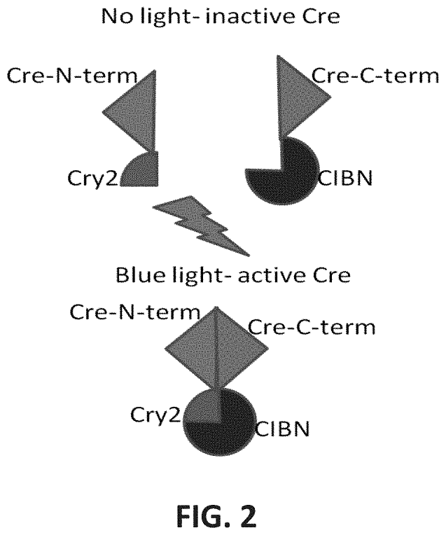

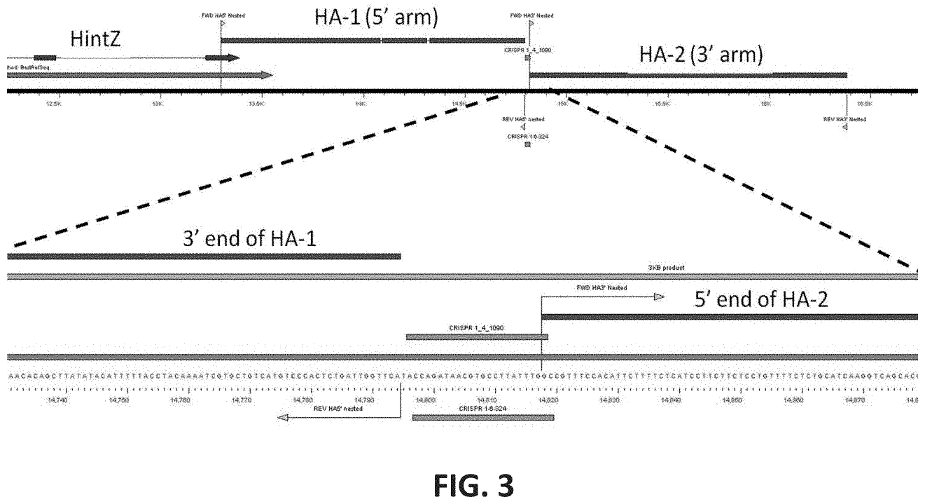

Generating a genome modified chicken line is a multi-step process. The final product is a female layer-hens line which is completely identical, with respect to genome content, to the layer which is used today in the industry Likewise, the end product which is the infertile egg used for food will be identical —see FIG. 1.

The workflow consists of 5 main steps:

1. Generating and cultivating chicken Primordial Germ-Cells (PGCs) lines.

2. Genome modification in cultured PGCs.

3. Transplantation of modified PGCs to embryos, and producing chimera chickens that will be screened for potential carriers.

4. Breeding founder chickens from genetic material obtained from the chimeras.

5. Expanding the founder chickens colonies to founder flocks and re-verifying the germline transmission.

### MATERIALS AND METHODS

PGC culture medium: Avian PGC culture medium consists of DMEM (Gibco) calcium free medium diluted with water to 250 mosmol/L, containing 12.0 mM glucose, 2.0 mM GlutaMax (Gibco), 1.2 mM pyruvate (Gibco), 1×MEM vitamin (Gibco), 1×B-27 supplement (Gibco), 1×NEAA (Gibco), 0.1 mM β-mercaptoethanol (Gibco), 1×nucleosides (Biological industries), 0.2% ovalbumin (Sigma), 0.1mg/ml sodium heparin (Sigma), CaC12 0.15mM (Sigma), 1×MEM vitamin (Gibco), 1×Pen/Strep (Biological industries), 0.2% chicken serum (Sigma) in avian DMEM. The following growth factors were added before use: human Activin A, 25 ng/mL (Peprotech); human FGF2 4 ng/mL (R&D Biosystems), ovotransferin (5μg/m1) (Sigma). AkoDMEM refers to a Diluted medium containing glucose, pyruvate and vitamins.

PGC line derivation: PGC lines were derived by placing ˜1.0-3.0 μL of blood isolated from stage 15 to 16 (H&H) embryos in 300 μL medium in a 48-well plate. Medium was changed every 2 days. When total cell number reached 1×10⁵, total volume of medium was changed every 2 days and cells were propagated at 2-4×10⁵ cells/ml medium. Cells were frozen in PGC culture medium containing 10% DMSO, temperature was gradually decreased to −80 ° C., stored for 1-3 days and transferred to liquid nitrogen.

Sex determination and PGC line characterization: Each PGC line was characterized for sexing, mRNA expression of PGC markers, and protein expression of the known PGCs marker SSEA1⁴. DNA from the donor embryo was isolated and kept for future reference. For sexing, DNA from 2-4×10⁵ PGCs cells was collected, re-suspended in tail buffer (102-T, Viagen) containing 100 μg/ml Proteinase K (Sigma) and incubated at 55° C. for 3 hours. The Proteinase K was inactivated at 85 ° C. for 45 minutes. PCR for sex determination was performed with primers from W chromosome that target female chromosome (P17, P18) and Ribosomal S18 (P19, P20) as a control⁶. For gene expression analysis, RNA was purified using TRIZOL reagent and 1 μg of RNA was used for cDNA library production by reverse transcription PCR reaction (GoScript Reverse transcriptase, Promega). The cDNA served as a template for PCR by using Daz1, Sox2, cPouV, Nanog, Klf4, cVH primers, P21-P22, P23-P24, P25-P26, P27-P28, P29-P30, P31-P32, respectively.

Immunohistochemistry with anti SSEA1 antibody: Cells were collected, fixed with 4% PFA, blocked with 5% normal goat serum in PBS 0.1% triton and stained at 1:100 dilution of anti-SSEA1 antibody (DSHB, Hybridoma bank¹³) in blocking buffer overnight. After washing the cells for 30min with PBS 0.1% triton secondary antibody was added (Alexa Fluor 488, molecular probes) for 1 hour, cells counterstained with DAPI (Sigma), mounted with mounting medium (Histomount, electron microscopy sciences) and covered.

PGCs transfection, selection and FACS sorting: Plasmid transfection of PGCs was done using lipofection or electroporation. For lipofection, Lipofectamine 2000 was used according to the manufacturer's protocol. 3-5×10⁵ cells were seeded in 96 well plate in AkoDMEM containing NEAA, pyruvate, vitamins, CaCl₂ and growth factors (activinA, hFGF and ovatransferin). 100 ng of plasmid, and 0.25 μl of Lipofectamine 2000 (invitrogen) were diluted separately in 20 μl of OPTI-MEM mix together, incubated for 20 minutes and pipetted on the cells. For electroporation, 5×10⁵ or 1.5×10⁶ cells were washed in AkoDMEM and electroporated at 1000V, 12 ms, 3 pulses on Neon electroporator (Invitrogen), and seeded immediately in 96 or 48 well plate, respectively, in antibiotics-free PGCs medium. Medium was changed after 1-3 hours. Selection with 25-100 μg/ml G418 started 72 hours later for 2-4 weeks. Following selection, cells were individually isolated manually or by FACS sorting. For FACS sorting, gentle cells pipetting was done and cells were sorted in PGCs culture medium. Positive GFP cells sorted with FACS Aria II to new 96 wells plate, a single cell per well, or pooled. (FACS analysis performed using a BD FACS Aria II flow cytometer (BD, USA).

### Plasmid Preparation

Cloning of the CRISPR plasmids: CRISPR sequences were design using CRISPR design tool, Zhang lab, MIT (www(dot)crispr(dot)mit(dot)edu)⁹. px330-GFP plasmid (modified from Addgene plasmid #42230)¹⁴ was cut using BbsI restriction enzyme and served us as the backbone for CRISPR site insertion to form the sgRNA. The oligos for the sgRNA CRISPR sites—CRISPR1, CRISPR3 (oligos P34-P35 and P36-P37, respectively) were denatured at 95 ° C. for 30 seconds, slowly annealed and ligated to the BbsI cut plasmids, transformed into E. coli, purified and sequence verified as described [mediadotaddgenedotorg/cms/filer_ public/e6/5a/e65a9ef8-c8ac-4f88-98da-3b7d7960394c/zhang-lab-general-cloning-protocoldotpdf; and Cong L, et al., Science. 2013 Jan. 3. 10.1126/science.1231143 PubMed 23287718].

Cloning of the pJet-HAs plasmid: The genomic region downstream to the HINTZ locus on the Z chromosome, containing both the 5′HA and 3′HA was amplified from PGCs DNA with P1 and P2 primers, using PCR (Kapa, Roche). PCR product was purified and ligated into pJet1.2 plasmid (Invitrogen) according to manufacturer protocol.

Construction of the targeting vector: The pCAGG-IRES-Neo-GFP plasmid was used as template for PCR, using P5-P6 primers, to amplify the insert pCAGG-IRES-Neo-GFP. pJet-HAs plasmid was used as template for PCR, using P3-P4 primers, to amplify the vector containing 5′HA and 3′HA (Illustration 3). Gibson assembly reaction was done to the purified vector and insert PCR products taking 0.03 pm, 0.06 pm linearized product, respectively. Gibson assembly reaction¹⁰ products were transformed to E. coli for plasmid preparation which was sequence verified.

### Construction of the pCAGG-Optogene Vector

To generate the pCAGG-Optogene vector the optogenes plasmids pmCherry-CIBN-CreC and pmCherry-Cry2-CreN¹¹ were used as a template to amplify the optogenes using the P40-P41 and P42-P43 primers, which yielded 1.3 kb and 2.1 kb products respectively. These two products share overlap sequences at the P2A site which was introduced in primers P41 and P42. Single-cycle o verhang extension PCR was used to unite to two fragments, to a single 3.5 kb product which was cleaned from an agarose gel. This product was ligated to pJet1.2 shuttle vector that was used as a temple for PCR using primers P44 and P45, which contain tails with SmaI and NheI restriction sites, respectively. This product was digested using the appropriate restriction enzymes and was used as an insert for ligation to ligated to SmaI and NheI digested pCAGG-IRES-GFP plasmid that served as a vector. The ligation products were transformed to E. coli bacteria and the propagated plasmid was sequenced verified.

### Construction of the pGK-DTA-IRES-GFP Vector

To generate the pGK-DTA-IRES-GFP, the expression vector pSK BS-PGK-DTA was used as a template for PCR with primers P46 and P47 which contain extensions sequences for the XmaI and NheI restriction sites respectively. The 0.65 kb product was digested with the respective enzymes and was used as an insert for ligation to the XmaI-NheI complementary site in the pGK-IRES-GFP plasmid that served as a vector for the ligation. The ligation products were transformed to E. coli bacteria and the propagated plasmid was sequenced verified.

In-ovo electroporation: In-ovo electroporation was conducted essentially as was previously described. Fertile eggs were incubated for 56-60 h at 37.8 ° C., the eggshell was windowed and plasmid DNA at a concentration of ˜2 μg/μl was injected using a sharpened micro-pipette with an opening of 10-15 μm in diameter to the neural tube. Three pulses of 25 V, 30 ms were delivered using ECM 830 square wave electroporation system (BTX). Following electroporation, the eggshell was sealed with parafilm and the embryos were further incubated until analysis.

Endonuclease assay: PGCs were transfected with CRISPR1 or CRISPR3 plasmids using Lipofectamine 2000 reagent. Forty-eight hours later, individual GFP positive cells were isolated into 96 well plate and grown to form pure colonies. DNA was collected and a 350 bp region flanking the CRISPR sites was PCR amplified with P38-P39 primers. The PCR products undergo denaturation at 95° C. and slowly annealed and incubated with T7 endonuclease for 1 h at 37° C. For calibration purposes and as a positive control, the 350 bp PCR product was sub-cloned to pJet1.2 and the CRISPR site was mutated using site-directed mutagenesis. The mutation that was introduced replaced the WT sequence ATACCAGATAACGTgCCTTATTTGGCCGTT (SEQ ID NO: 2) with ATACCAGATAACGTaatCCTTATTTGGCCGTT (SEQ ID NO: 3). This artificial mutation served as a positive control to both the endonuclease assay (FIG. 7A) and for control sequencing (FIG. 8B).

Southern blot assay: Dig-labeling for the 5′HA, 3′HA and Neo gene probes were prepared by PCR amplification (Longamp, NEB) with primers P13-P14, P15-P16 and P11-P12, respectively, using DIG DNA labeling Mix (Roche). 15 μg of genomic DNA were digested overnight at 37 ° C. with BglII restriction Enzyme. DNA fragments were separated by electrophoresis on 0.8% (w/v) agarose gel (20 V, 12 h) and transferred onto positively charged nylon membranes (GE Healthcare). Following transfer, humid membranes were cross linked using a UV light set to 254 nm for 3 minutes on each side then rinsed with 2×SSC. Membranes were pre-hybridized for 2 hours at 42° C. using DIG Easy-Hyb hybridization solution (Roche). Probes (50 ng/ml) were denatured by heating to 95° C. for 5 minutes and immediately plunged into ice. Denatured probes were added to 10 ml warm DIG Easy-Hyb solution and hybridized for 12 hours at 42° C. Membranes were washed twice for 10 minutes in 2×SSC, 0.1% SDS at room temperature under agitation and then washed 3 times for 30 minutes in 0.2×SSC, 0.1% SDS at 65° C. under agitation. Further washing and blocking was done with a DIG wash and block buffer set (Roche) and according to their protocol. DIG labeling was detected using Anti-Digoxigenin-AP antibody 1:10000 (Roche) followed by chemiluminescence reaction using CDP-Star reagent (Roche). Images were taken using G:BOX gel imaging system (Syngene).

PGC injection to embryos and whole mount staining: Freshly laid eggs were incubated with the pointed end up for 58-62 hours at 37.8° C. with 55% humidity. Following incubation, 4-8 mm window was opened in the egg shell and 3000-8000 PGCs were injected to the blood stream using sharpened micropipette with opening of ˜30-40 μm. The window was covered with white egg membrane and further sealed with a Parafilm (Parafilm) or a Leukoplast (BSN medical GmbH) tape. Embryos were incubated until hatching. Some gonads of injected embryos were isolated and taken for whole-mount GFP staining. Gonads were fixed in 4% PFA, washed for 2 h with PBS blocked with 5% normal donkey serum in PBS 1% Triton and stained at 1:20 dilution of mouse anti-SSEA1 antibody¹³ or rabbit anti GFP antibody 1:500 (Abcam) in blocking buffer overnight. After washing for 2 h with PBS 1% triton, a secondary donkey anti mouse cy3 antibody 1:500 was added (Jackson Immunoresearch laboratories) or secondary alexa488 anti rabbit antibody 1:500 (Molecular Probes) for 3 hours in blocking buffer. Tissue counterstained with DAPI (Sigma) and mounted in glycerol, and imaged by confocal microscope (Leica, TCS SPE, Wetzlar, Germany).

TABLE 3
List of primers

| primer | Sequence |
|---|---|
| P1 | TTTTGAATGAAGGGCCTGAG SEQ ID NO: 4 |
| P2 | TGAACCAATCAGAGTGGGAC SEQ ID NO: 5 |
| P3 | GTCCCTCTTCTCTTATGGAGATCGCCGTTTCCACATTCTTTTCTC SEQ ID NO: 6 |
| P4 | GGTGGCACTTTTCGGGGAAATGTGTGAACCAATCAGAGTGGGACATGAC SEQ |
| | ID NO: 7 |
| P5 | GTCATGTCCCACTCTGATTGGTTCACACATTTCCCCGAAAAGTGCCACC SEQ |
| | ID NO: 8 |
| P6 | GAGAAAAGAATGTGGAAACGGCGATCTCCATAAGAGAAGAGGGAC SEQ ID |
| | NO: 9 |
| P7 | GAAGTGTGCTGCTAACCTG SEQ ID NO: 10 |
| P8 | GCTATGAACTAATGACCCCG SEQ ID NO: 11 |
| P9 | TTTTCCTCCTCTCCTGACTAC SEQ ID NO: 12 |
| P10 | GGCCTGGATGATAAGAGTCTTC SEQ ID NO: 13 |
| P11 | GCTATTCGGCTATGACTGGG SEQ ID NO: 14 |
| P12 | GAAGGCGATAGAAGGCGATG SEQ ID NO: 15 |
| P13 | GTGGAACACAGCTTTTCCAG SEQ ID NO: 16 |
| P14 | GCTCTTCAACTTGCCATTTG SEQ ID NO: 17 |
| P15 | TCAACAGCACGTAAGCAAC SEQ ID NO: 18 |
| P16 | CCTGACTCCATTTTTGAGCC SEQ ID NO: 19 |
| P17 | CCCAAATATAACACGCTTCACT SEQ ID NO: 20 |
| P18 | GAAATGAATTATTTTCTGGCGAC SEQ ID NO: 21 |
| P19 | AGCTCTTTCTCGATTCCGTG SEQ ID NO: 22 |
| P20 | GGGTAGACACAAGCTGAGCC SEQ ID NO: 23 |
| P21 | CAACTATCAGGCTCCACCAC SEQ ID NO: 24 |
| P22 | CTCAGACGGTTTTCAGGGTT SEQ ID NO: 25 |
| P23 | AGGCTATGGGATGATGCAG SEQ ID NO: 26 |
| P24 | GTAGGTAGGCGATCCGTTCA SEQ ID NO: 27 |
| P25 | CGAGACCAACGTGAAGGGAA SEQ ID NO: 28 |
| P26 | CAGACCCGGACAACGTCTTT SEQ ID NO: 29 |
| P27 | CTCTGGGGCTCACCTACAAG SEQ ID NO: 30 |
| P28 | AGCCCTGGTGAAATGTAGGG SEQ ID NO: 31 |
| P29 | AGCTCTCATCTCAAGGCACA SEQ ID NO: 32 |
| P30 | GCIAAACIATCCACTGCTTCA SEQ ID NO: 33 |
| P31 | AGCACAGGTGGTGAACGAACCA SEQ ID NO: 34 |
| P32 | TCCAGGCCTCTTGATGCTACCGA SEQ ID NO: 35 |
| P34 | CACCGCCAAATAAGGCACGTTATC SEQ ID NO: 36 |
| P35 | AAACGATAACGTGCCTTATTTGGC SEQ ID NO: 37 |
| P36 | CACCGACCAGATAACGTGCCTTATT SEQ ID NO: 38 |
| P37 | AAACAATAAGGCACGTTATCTGGT SEQ ID NO: 39 |
| P38 | TTGCAGTGGTTACCGTTCG SEQ ID NO: 40 |
| P39 | TAGTAGGCATCTTGTGGGGG SEQ ID NO: 41 |
| P40 | ATGAATGGAGCTATAGGAGG SEQ ID NO: 42 |
| P41 | CCACGTCTCCTGCTTGCTTTAACAGAGAGAAGTTCGTGGCATCGCCATCTTCC |
| | AGCAGGCG SEQ ID NO: 43 |
| P42 | TGTTAAAGCAAGCAGGAGACGTGGAAGAAAACCCCGGTCCTATGAAGATGG |
| | ACAAAAAGAC SEQ ID NO: 44 |
| P43 | TTACAGCCCGGACCGACGATG SEQ ID NO: 45 |
| P44 | ATCTGACCCGGGATGAATGGAGCTATAGGAGG SEQ ID NO: 46 |
| P45 | GTAGCTGCTAGCTTACAGCCCGGACCGACGATG SEQ ID NO: 47 |
| P46 | CAGGTCCCCGGGATGGATCCTGATGATGTTG SEQ ID NO: 48 |
| P47 | GCATGTGCTAGCTTAGAGCTTTAAATCTCTG SEQ ID NO: 49 |

```
Plasmid Sequences
  1. pX330-GFP (SEQ ID NO: 50)
  2. CRISPR1 (SEQ ID NO: 51)
  3. CRISPR3 (SEQ ID NO: 52)
  4. pJet-Has (SEQ ID NO: 53)
  5. pCAGG-Neo-IRES-GFP (SEQ ID NO: 54)
  6. Targeting Vector (SEQ ID NO: 55)
  7. pmCherry-Cry2-CreN (SEQ ID NO: 56)
  8. pmCherry-CIBN-CreC (SEQ ID NO: 57)
  9. pB-RAGE-GFP (SEQ ID NO: 58)
10. pCAGG-IRES-GFP (SEQ ID NO: 59)
11. pCAGG-Optogenes (SEQ ID NO: 60)
12. pB-RAGE-mCherry (SEQ ID NO: 61)
13. pSK BS-PGK-DTA (SEQ ID NO: 62)
14. pGK-IRES-GFP (SEQ ID NO: 63)
15. pGK-DTA-IRES-GFP (SEQ ID NO: 64)
```

### RESULTS

### PGC Lines Derivation and Characterization

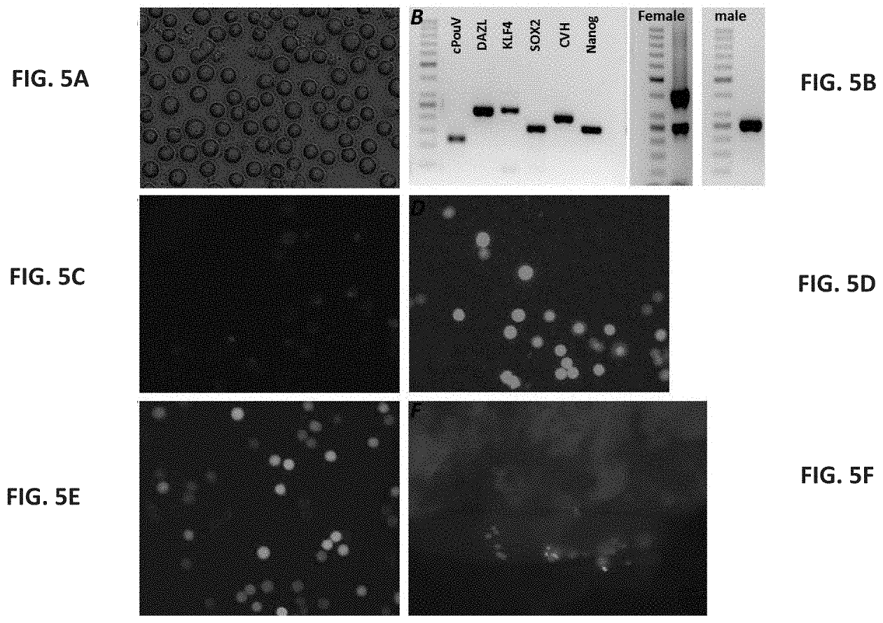

During the earliest stages of embryonic development, soon after oviposition and prior to the initiation of gastrulation, PGCs migrate rostrally to the Germinal Crescent region at the anterior part of the extra-embryonic mesoderm layer. It is thought that this migration “protects” the PGCs from undergoing differentiation processes as somatic cells do. It is not until the formation of the Area Opaca Vasculosa, blood and heart-beat, after about 2.5 days of incubation (Stage 14-17 H&H¹), that the cells return to the embryo through the blood stream and colonize the Genital Ridge which will give rise to the gonads. At these stages, using a micropipette with a ˜40-60 μm diameter opening, 1-3 μl of blood was collected from the vasculature system of the embryos and transferred into a well containing PGC culture medium in a 48 well plate. The PGC culture medium allows for fast division of the PGCs (20-24 hours of cell cycle) while retaining their undifferentiated state under feeder free conditions. After 2-3 weeks in culture, blood cells degraded and disappeared². Within another 1-2 more week, the cultured PGCs became confluent (FIG. 5A). These cells can be further grown for gene modification, or can be successfully frozen and thawed for latter modifications. Chicken PGCs in culture have been extensively characterized in the literature using morphological features, protein and mRNA expression patterns and finally by their ability of gonad migration when injected back into the vasculature of a stage-matched recipient embryo³⁻⁵. These characteristics were examined in the produced PGC cell cultures to show that they keep the well-established PGC features. Morphologically, the PGCs are big, slightly granulated cells about 15-20 μm diameter containing large nuclei. The PGCs are totipotent cells, thus they express pluripotent markers such as the cPouV, SOX2, KLF4 and Nanog) and two unique germ cells markers—cVH and DAZL. For each PGC line, DNA was extracted for sex determination using primers for the Ribosomal S18 (P19-P20, 256 bp product size) as a positive control and primers for the W chromosome (P17-P18, 415bp product size) to identify females (FIG. 5B)⁶. Additionally, the PGCs express the membrane SSEA-1 antigen⁴ (FIG. 5C).

10 lines of PGCs were established from layers and broilers, both male and female lines. Plasmid transfection was carried out using cationic-lipid transfection reagent Lipofectamine 2000 which interacts with the negatively charged DNA, allowing its penetration into the cell. Transfecting with GFP encoding plasmid (pCAGG-GFP⁷) resulted in about 15-20% transfection efficiency (FIG. 5D). Further, transfection efficiency of PGCs using electroporation, resulted in higher efficiency of up to 90% (FIG. 5E). To demonstrate that cultured PGCs successfully colonize the gonads, GFP-expressing PGCs were injected into the blood stream of stage 14-16 H&H and the embryos were incubated for 10 days. The embryos were dissected and GFP-positive cells were identified in the gonads (FIG. 5F).

### Designing the CRISPR-Cas9 Targets on the Z Chromosome

DNA editing into the Z chromosome were done using CRISPR-Cas9 and homologous recombination processes. While CRISPR-Cas9 system will directly cut the DNA at a specific site of the Z chromosome, the endogenous repair system using homology recombination process will allow targeted insertion of the desired DNA into the precise location. For this purpose, constructing a targeting vector plasmid which contains the homology arms corresponding to the insertion site on the Z chromosome is required. The site for DNA insertion at the Z chromosome downstream the coding gene HINTZ was chosen. The use of the CRISPR system has been shown in many studies to improve direct DNA insertion events. Extensively used for that purpose, the px330 plasmid includes the sgRNA site and the Cas9 enzyme⁸. The sgRNA site contains a unique sequence which directs the Cas9 enzyme to the target site and leads to specific genome targeted DSDB. Using a CRISPR design engine tool, a unique sequence for the sgRNA was identified as shown in FIG. 6A. The top 12 guides, according to their score are depicted in FIG. 6B and in Table 1.

Twenty nucleotide sequences—guide #1 and #3 were chosen by conventional similarities of the secondary structure and by checking possible off-target sites in the chicken genome which scored by the mismatch extent. The top 10 results of search for potential off-targets for guide #1 are shown in FIG. 6C and in Table 2. Notably, the top 6 off-targets have 4 mismatches, highlighting the specificity of this guide.

DNA sequence insertion was carried out by cutting a modified px330 plasmid, which contains in-frame GFP fused to the c-terminus of Cas-9. Annealed primers containing the sgRNA sequences were ligated to the BbsI restriction enzyme as previously described. (FIG. 6B). Ligation products were transformed to E. coli, plasmids were purified and sgRNA insertions were verified by sequencing.

### Activity Validation of the CRISPR-Cas9 System

By growing PGCs in feeder free culture medium, pure colonies originating from single cells were obtained, thereby allowing characterization of the efficiency of the CRISPR-Cas9 system. To this end PGCs were transfected with either pX330-GFP-CRISPR1 and pX330-GFP CRISPR3 plasmids, and clonal colonies were grown. Total genomic DNA was extracted from colonies originating from single cells expressing GFP. The DNA was analysed by endonuclease assay and sequenced. For the endonuclease assay, a positive control was designed. This control was a 320 bp PCR product with inserted mutations at the predicted site for CRISPR-Cas9 activity. This product was mixed with similar length WT product in different ratios, 1:15, 1:7, 1:1—mutated:WT respectively, and the anealed mixture was subjected to endonuclease activity (FIG. 7A). Two short bands at the predicted size of 136 bp and 184 bp were clearly visible at the ratios of 1:7 and 1:1, indicating the assay was properly working Similarly, the same assay was performed on genomic DNA obtained from 12 colonies, transfected with either CRISPR1 and CRISPR3 plasmids (FIGS. 7B, C). In 9 out of the 12 colonies, a clear doublet at the predicted size was observed. This indicates that both CRISPR1 and CRISPR3 plasmids, efficiently generate DSDBs at the predicted site.

For sequencing analysis, PCR products that were used for endonculease assay (FIGS. 7A-C), were also sequenced (FIGS. 8A-D). Sequencing of the WT negative control revealed the predicted cleavage site of CRISPR1 (FIG. 8A). Sequencing a mixture of WT and artificially mutated product, as a positive control, revealed the appearance of double peaks on the DNA chromatogram, immediately after the predicted cleavage site (Blue arrowhead, FIG. 8B). Similar sequencing of the same genomic region in the transfected colonies revealed both negative (FIG. 8C) and positive (blue arrowhead, FIG. 8D) colonies, whereas the latter were >70% of the cases.

### Constructing the Targeting Vector for Genome Integration

To demonstrate a targeted genomic integration to the Z chromosome, using HR, a targeting vector was designed (FIGS. 9A-F). The vector contains a pCAGG promoter followed by the neomycin selections gene, internal ribosome entry site (IRES), GFP and the Rabbit beta-globin polyadenylation site. This cassette was flanked by ˜1.5 kb homology arms at the 5′ and 3′ ends respectively. To generate this vector, a ˜3 kb DNA fragment, containing both homology arms was amplified using primers P1 and P2, and ligated to a shuttle vector pjet1.2. Full sequencing of this fragment was found to be identical to the chicken genome sequence. This plasmid—pJet-HAs, was used as a template to generate a linearized PCR product containing two separated homology arms excluding a 23 bp sequence between them, which contains the CRISPR sgRNA sites. The amplification was done using the P3 and P4 primers which contain sequences, at their 5′ end, which correspond to the edges of the pCAGG-Neo-IRES-GFP cassette. This linear PCR product is referred to as the “vector”. The pCAGG-Neo-IRES-GFP plasmid was used as a template to generate a linear PCR product. This fragment was amplified using primers P5 and P6 containing sequences which correspond to the 3′ and 5′ ends of the 5′HA and 3′HA ends, respectively. This product is referred to as the “insert”. The vector and the insert were stitched together using the Gibson assembly reaction¹⁰, to create the final targeting vector.

### Homologous Recombination to the Z Chromosome Using the Targeting Vector and CRISPR Plasmids

The ability to obtain pure PGCs colonies from a single cell enables the identification of positive colonies that underwent correctly inserted HR, using methods such as PCR and Southern blot. For PGC transfection, lipofection with a 5-10% (FIG. 10A) transfection efficiency or electroporation with >40% efficiency were used. Transfection was carried out with two plasmids, the targeting vector and one of the two CRISPR plasmids described above (CRISPR1 or CRISPR3). Following the transfection, the cells were left to recover for 24 hours, and transferred to G-418 containing media, for selection. After two weeks of selection, only G-418 resistant cells survived, of them >99% were GFP positive (FIG. 10B). To verify that the cells retain their ability to colonize the gonads, they were injected into host embryos as was described above in FIG. 1F (FIG. 8C). The gonads were immuno-stained with anti-GFP antibody and the colonization of GFP-positive PGCs cells in the gonads was verified using confocal microscope (FIG. 10D).

The G-418 resistant, GFP-positive cells consist of a potentially heterogeneous population. Thus, in order to verify the HR integration, and to obtain pure homogenous population, single GFP-positive cells were separated using FACS sorting to 96 well plate (FIG. 11A). Pure colonies were raised and genomic DNA was extracted for PCR and Southern blot analysis. In parallel, pooled GFP-positive cells were FACS sorted. For PCR analysis, two sets of primers were designed. The first, forward P7 upstream to the 5′HA and reverse P8 from the CAGG promoter (1.6 kb product size), and the second, forward P9 from the rabbit beta-globin polyadenylation site and reverse P10 downstream to the 3′HA (1.8 kb product length, FIG. 11B). Both in pooled cells (FIG. 11C) and in pure colonies (FIG. 11D), the expected products for the 5′ and 3′ were detected, indicating that correct HR integration had occur in these cells.

To further verify the correct HR integration as well as to confirm that only a single copy of the targeting vector was integrated into the genome, a Southern blot analysis was conducted. Two PGCs cell lines from male and female donors were analyzed. Notably, the female line has only a single copy of the Z chromosome. Three dig-labeled DNA probes were designed (yellow boxes in FIG. 12A and FIG. 12B). The first two probes, amplified using primers P11-P12 and P13-P14, 500 bp long each, are located upstream and downstream to the 5′ and 3′ HAs respectively. The third probe, amplified using primers P15-P16, 704bp long, is designed to detect the Neo gene inside the targeting vector, thus it allows for confirmation that only a single copy of the vector was integrated. The BglII restriction enzyme was used to cleave the genomic DNA for analysis. Two restriction sites, ˜6.5 kb apart from each other, are located on the WT chromosome, upstream and downstream to the 5′ and 3′ probes respectively. Additional BglII site is located in targeting vector, yielding a predicted 7.5 kb and 3.3 kb fragments to identify correct HR integration. The results of the Southern blot analysis on the genomic DNA extracted from the male PGCs line revealed 2 bands at the predicted size, 6.5 kb for the WT allele and 7.5 kb and 3.3 kb for the allele which underwent correct HR integration, for the 5′ and 3′ sites, respectively. This was confirmed for both the DNA from pooled cells as well as for pure colonies (FIG. 12C). A similar analysis was carried out for the female PGC cell line. In this case a single band at the predicted size of 7.5 kb for the 5′ integration site was found. As the female genome contains only a single copy of the Z chromosome, no WT allele (6.5 kb) was detected. Probing the Neo gene, revealed a single band at the predicted size of 7.5 kb, confirming that only a single copy of the targeting vector was integrated into the genome (FIG. 12D).

### Validation of the Optogenetic System in HEK293 Cells In-Vitro and in Chicken Embryos In-Ovo

To verify the activity of the inducible system in-vitro and in chicken embryos in-ovo, three plasmids: pmCherry-Cry2-CreN, pmCherry-CIBN-CreC and the reporter PB-RAGE-GFP were transfected to HEK293 cells (FIG. 13) and to chick embryos (FIG. 14). The first two optogenic plasmids encode the reporter gene mCherry which confirms successful transfection. The PB-RAGE-GFP expression vector contains a multiple stop codons sequence flanked by LoxP sites upstream to the GFP coding region. Upon Cre activation, the STOP codons are removed thus allowing the GFP to be expressed. While in negative-control HEK293 cells that were triple-transfected and kept in the dark, there were no GFP-positive cells (FIG. 13, upper row). In cells that were exposed to blue-light illumination, 24 hours after transfection, many cells expressed GFP (FIG. 13, lower row), confirming the activation of the optogenetic system in these cells.

To verify the activity of the optogenetic system in-ovo, a triple transfection with pmCherry-Cry2-CreN, pmCherry-CIBN-CreC and PB-RAGE-GFP plasmids by electroporation into chicken embryo neural tubes at stage 16 H&H was performed. Twelve hours following electroporation, experimental group embryos were subjected to 15 seconds of blue-light illumination, while negative control embryos were kept in the dark. The embryos were incubated for an additional 12 hours and checked for GFP expression under fluorescent stereoscope (FIG. 14). While in embryos that were kept in dark (FIG. 14, upper row), only mCherry was expressed—confirming successful electroporation, in embryos of the experimental group, GFP positive cells were clearly evident (FIG. 9, lower row), confirming that the light-inducible Cre was activated.

The optogene plasmids pmCherry-Cry2-CreN and pmCherry-CIBN-CreC drive the expression of the genes using the CMV promoter¹¹ which is unfavorable in chicken cells. To overcome this and to combine the two to a single vector, the present inventors designed a plasmid vector which drives the expression of CIBN-CreC and Cry2-CreN, linked by the P2A self-cleaving peptide, followed by IREG-GFP, under the CAGG promoter, which is highly active in chicken cells. Synthesis of pCAGG-CIBN-CreC-P2A-Cry2-CreN-IRES-GFP was based on a modification of the original optogenes plasmids described in Kennedy et al¹¹. Each of these plasmids encode mCherry followed by IRES sequence with either CIBN-CreC (a truncated form of CIB1 fused to the C-terminal of the Cre enzyme) or CRY2-CreN (Cryptochrome 2 fused to the N-terminal of the Cre enzyme. FIG. 15A). The goal of the following cloning was to join the two fusion optogenes with self-cleaving peptide P2A, under the CAGG promoter, followed by IRES-GFP. To this end, the CIBN-CreC plasmid was used as a template for PCR with P40 and P41 primers and the CRY2-CreN plasmid was used as a template for PCR with P42 and P43 primers (FIG. 15A). Notably primers P41 and P42, which contain the P2A cleavage site, share overlap sequence that allows the two products to be merge by a single-cycle overhang extension PCR (FIG. 15B). This product which contains CIBN-CreC-P2A- CRY2-CreN was ligated to a shuttle vector pJet1.2, that was sequences verified (FIG. 15C). This plasmid served as template for PCR with primers P44 and P45 which added to the product the SmaI and NheI restriction site on the 5′ and 3′ ends, respectively (FIG. 15D). This product was digested with the restriction enzymes and ligated to the pCAGG-IRES-GFP plasmid which also cut using the same enzymes (FIG. 15E). This ligation product contains the CAGG promoter, followed by CIBN-CreC, P2A self-cleaving peptide, Cry2-CreN, IRES, GFP and the rabbit beta-globin poly-adenylation site (referred to herein as pCAGG-Optogenes), was sequenced verified (FIG. 15F).

To verify the activity of the pCAGG-Optogene vector in-vitro, the plasmid, which expresses GFP as a reporter for successful transfection, was co-transfected into HEK293 cells with pB-RAGE-mCherry Like the the PB-RAGE-GFP vector described above (FIG. 13), the pB-RAGE-mCherry contains a multiple stop codon sequence flanked by LoxP sites upstream to the mCherry coding region. Upon Cre activation, the STOP codons are removed thus allowing the mCherry to be expressed (FIG. 16). While in HEK293 cells that were co-transfected and kept in the dark there were no mCherry-positive cells (FIG. 16, upper row), in cells that were exposed to blue-light illumination, many cells were expressing mCherry (FIG. 16, lower row), confirming that the single-vector strategy of the pCAGG-Optogenes, preserves the optogenic properties of the system.

To verify the activity of the pCAGG-Optogenes vector in living chick embryos in-ovo, the plasmid was co-transfected by electroporation to stage 14-16 H&H chick embryos together with pB-RAGE-mCherry. Twelve hours following electroporation, negative-control group eggs were kept in the dark, while experimental group embryos were exposed to blue-light for 15 seconds (FIG. 17). Both groups were further incubated for 12 h and examined under a fluorescent stereoscope. Following incubation, both groups revealed high level of GFP expression, indicating the successful electroporation. However, only in the light-exposed group (FIG. 17, lower row), mCherry-positive expressing cells were identified, indicating that the optogenic system, using the single-vector strategy of the pCAGG-Optogenes, was activated in a light-inducible manner.

### Inducing Lethality in Chick Embryos

To demonstrate the feasibility of causing mortality using a toxin, the coding region of the DTA¹², commonly used as negative selection marker, was cloned into an expression vector containing the pGK promoter followed by an IRES GFP (pGK-IRES-GFP). This plasmid also served as a negative control. The DTA coding region was cloned upstream to the IRES sequence giving rize to the pGK-DTA-IRES-GFP, which upon expression in cells inhibits protein synthesis which leads to cellular death.

To test the effects of DTA expression in chicken embryos, stage 14-16 H&H embryos were electroporated with either pGK-IRES-GFP, as a negative control or with pGK-DTA-IRES-GFP vector. Twelve hours following electroporation, the embryos were analysed for the expression of GFP under a fluorescent microscope (FIG. 18). While in control embryos, GFP was widely expressed in the neural tube (FIG. 18), in DTA expressing embryos, no GFP expression was detected, indicating that protein synthesis was blocked in these cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. HAMBURGER, V. & HAMILTON, H. L. A series of normal stages in the development of the chick embryo. J. Morphol. 88, 49-92 (1951).
2. Nandi, S. et al. Cryopreservation of specialized chicken lines using cultured primordial germ cells. Poult. Sci. 95, 1905-1911 (2016).
3. van de Lavoir, M.-C. et al. Germline transmission of genetically modified primordial germ cells. Nature 441, 766-9 (2006).
4. Karagenç, L., Cinnamon, Y., Ginsburg, M. & Petitte, J. N. Origin of primordial germ cells in the prestreak chick embryo. Dev. Genet. 19, 290-301 (1996).
5. Naito, M., Harumi, T. & Kuwana, T. Long-term culture of chicken primordial germ cells isolated from embryonic blood and production of germline chimaeric chickens. Anim. Reprod. Sci. 153, 50-61 (2014).
6. Clinton, M., Haines, L., Belloir, B. & McBride, D. Sexing chick embryos: a rapid and simple protocol. Br. Poult. Sci. 42, 134-8 (2001).
7. Cinnamon, Y., Ben-Yair, R. & Kalcheim, C. Differential effects of N-cadherin-mediated adhesion on the development of myotomal waves. Development 133, 1101-12 (2006).
8. Cong, L. et al. Multiplex genome engineering using CRISPR/Cas systems. Science 339, 819-23 (2013).
9. Ran, F. A. et al. Genome engineering using the CRISPR-Cas9 system. Nat. Protoc. 8, 2281-2308(2013).
10. Gibson, D. G. et al. Enzymatic assembly of DNA molecules up to several hundred kilobases. Nat. Methods 6, 343-345 (2009).
11. Kennedy, M. J. et al. Rapid blue-light?mediated induction of protein interactions in living cells. Nat. Methods 7, 973-975 (2010).
12. Lu, Q. R. et al. Common developmental requirement for Olig function indicates a motor neuron/oligodendrocyte connection. Cell 109, 75-86 (2002).
13. Solter, D. & Knowles, B. B. Monoclonal antibody defining a stage-specific mouse embryonic antigen (SSEA-1). Proc. Natl. Acad. Sci. U. S. A. 75, 5565-9 (1978).
14. Mashiko, D. et al. Feasibility for a large scale mouse mutagenesis by injecting CRISPR/Cas plasmid into zygotes. Dev. Growth Differ. 56, 122-9 (2014).

What is claimed is:

1-40. (canceled)

41. A DNA editing agent comprising a first nucleic acid sequence for eliciting in an inducible manner a lethal phenotype of a male chick embryo in an egg of a bird and a second nucleic acid sequence for directing said first nucleic acid sequence to a Z chromosome of a cell of the bird, said second nucleic acid sequence comprises
(i) a left homology arm (LHA) comprising nucleotide sequence that is substantially homologous to a 5′ region flanking a target gene locus in the Z chromosome of the bird; and
(ii) a right homology arm (RHA) comprising nucleotide sequence that is substantially homologous to a 3′ region flanking said target gene locus in the Z chromosome of the bird.

42. The DNA editing agent of claim 41, wherein said first nucleic acid sequence comprises
(i) sequence encoding an endonuclease enzyme which is operatively linked to a nucleotide sequence encoding a switch that controls the expression of said endonuclease enzyme, said switch being regulated by an inducer; and
(ii) sequence encoding a lethality protein, or sequence encoding a guide RNA that targets an essential gene of the bird, said lethality protein or said guide RNA is operatively linked to the activity of said endonuclease enzyme.

43. The DNA editing agent of claim 42, wherein said lethality protein is selected from the group consisting of a toxin, a pro-apoptotic protein, an inhibitor of the Wnt signaling pathway, a BMP antagonist and an FGF antagonist.

44. The DNA editing agent of claim 42, wherein said inducer is electromagnetic energy.

45. The DNA editing agent of claim 44, wherein said electromagnetic energy is a component of visible light.

46. The DNA editing agent of claim 45, wherein the component of visible light is blue light.

47. The DNA editing agent of claim 42, wherein said switch comprises a split recombinase enzyme that combines to form an active enzyme in the presence of said inducer.

48. The DNA editing agent of claim 42, wherein said essential gene is selected from the group consisting of BMPR1A, BMP2, BMP4 and FGFR1.

49. The DNA editing agent of claim 42, wherein said first nucleic acid sequence further comprises sequence encoding a RNA-guided DNA endonuclease enzyme.

50. The DNA editing agent of claim 49, wherein said RNA-guided DNA endonuclease enzyme is selected from the group consisting of zinc-finger nucleases (ZFNs), transcription activator-like effector nucleases (TALENs) and caspase 9.

51. The DNA editing agent of claim 41, wherein said bird is selected from the group consisting of chicken, turkey, duck and quail.

52. A cell population comprising cells of a bird, wherein said cells comprise the DNA editing agent of claim 41 for eliciting a lethal phenotype in a male offspring of the bird.

53. The cell population of claim 52, wherein said cells comprise primordial germ cells (PGCs) or gametes.

54. The cell population of claim 53, wherein said PGCs are selected from the group consisting of gonadal PGCs, blood PGCs and germinal crescent PGCs.

55. A method of generating a chimeric bird, said method comprises administering a population of PGCs to a recipient bird embryo under conditions sufficient to allow at least one of the PGCs to colonize a gonad of the recipient bird embryo, said PGCs comprise the DNA editing agent of claim 41, thereby generating a chimeric bird.

56. The method of claim 55, wherein said administering is by in-ovo injection.

57. A chimeric bird generated according to the method of claim 55.

58. A method of reducing the number of male chicks hatching from fertilized eggs of a bird, said method comprises:
introducing the DNA editing agent of claim 41 to a chick embryo in an egg of the bird for eliciting in an inducible manner a lethal phenotype in a male chick embryo in the egg; and
exposing said egg to an inducer which elicits the lethal phenotype, thereby reducing the number of male chicks hatching from the fertilized eggs of the bird.

59. The method of claim 58, wherein said bird is selected from the group consisting of chicken, turkey, duck and quail.

60. A method of reducing the number of male chicks hatching from fertilized eggs of a bird, said method comprises:
mating a female bird with a male bird, wherein a first exogenous polynucleotide which is operatively linked to a recombinase recognition site is stably integrated into a Z chromosome of said male bird, said first exogenous polynucleotide is for eliciting a lethal phenotype in a male chick embryo in an egg of the bird, and a second exogenous polynucleotide encoding a recombinase enzyme is stably integrated into a Z chromosome of said female bird, or
mating a female bird with a male bird, wherein a first exogenous polynucleotide which is operatively linked to a recombinase recognition site is stably integrated into a Z chromosome of said female bird, said first exogenous polynucleotide is for eliciting a lethal phenotype in a male chick embryo in an egg of the bird, and a second exogenous polynucleotide encoding a recombinase enzyme is stably integrated into a Z chromosome of said male bird,
wherein a lethal phenotype is induced in male chick embryo in an egg of the bird, thereby reducing the number of male chicks hatching from the fertilized eggs of the bird.


SEQUENCE LISTING (87 sequences)

SEQ ID NO: 1 - DNA, 149 bp, Artificial sequence
  Nucleic Acid sequence of the region between the two homology arms depicted in Fig 3
aacacagcttatatacatttttacctacaaaatcgtgctgtcatgtcccactctgattggttcataccagataacgtgccttatttggccgtttccacattcttttctcatccttcttctcctgttttctctgcatcaaggtcagcacg

SEQ ID NO: 2 - DNA, 30 bp, Artificial sequence
  wt nucleic acid sequence of the area flanking the CRISPR cleavage site
ataccagataacgtgccttatttggccgtt

SEQ ID NO: 3 - DNA, 32 bp, Artificial sequence
  mutated nucleic acid sequence of the area flanking the CRISPR cleavage site
ataccagataacgtaatccttatttggccgtt

SEQ ID NO: 4 - DNA, 20 bp, Artificial sequence
  Single strand DNA oligonucleotide
ttttgaatgaagggcctgag

SEQ ID NO: 5 - DNA, 20 bp, Artificial sequence
  Single strand DNA oligonucleotide
tgaaccaatcagagtgggac

SEQ ID NO: 6 - DNA, 45 bp, Artificial sequence
  Single strand DNA oligonucleotide
gtccctcttctcttatggagatcgccgtttccacattcttttctc

SEQ ID NO: 7 - DNA, 49 bp, Artificial sequence
  Single strand DNA oligonucleotide
ggtggcacttttcggggaaatgtgtgaaccaatcagagtgggacatgac

SEQ ID NO: 8 - DNA, 49 bp, Artificial sequence
  Single strand DNA oligonucleotide
gtcatgtcccactctgattggttcacacatttccccgaaaagtgccacc

SEQ ID NO: 9 - DNA, 45 bp, Artificial sequence
  Single strand DNA oligonucleotide
gagaaaagaatgtggaaacggcgatctccataagagaagagggac

SEQ ID NO: 10 - DNA, 19 bp, Artificial sequence
  Single strand DNA oligonucleotide
gaagtgtgctgctaacctg

SEQ ID NO: 11 - DNA, 20 bp, Artificial sequence
  Single strand DNA oligonucleotide
gctatgaactaatgaccccg

SEQ ID NO: 12 - DNA, 21 bp, Artificial sequence
  Single strand DNA oligonucleotide
ttttcctcctctcctgactac

SEQ ID NO: 13 - DNA, 22 bp, Artificial sequence
  Single strand DNA oligonucleotide
ggcctggatgataagagtcttc

SEQ ID NO: 14 - DNA, 20 bp, Artificial sequence
  Single strand DNA oligonucleotide
gctattcggctatgactggg

SEQ ID NO: 15 - DNA, 20 bp, Artificial sequence
  Single strand DNA oligonucleotide
gaaggcgatagaaggcgatg

SEQ ID NO: 16 - DNA, 20 bp, Artificial sequence
  Single strand DNA oligonucleotide
gtggaacacagcttttccag

SEQ ID NO: 17 - DNA, 20 bp, Artificial sequence
  Single strand DNA oligonucleotide
gctcttcaacttgccatttg

SEQ ID NO: 18 - DNA, 19 bp, Artificial sequence
  Single strand DNA oligonucleotide
tcaacagcacgtaagcaac

SEQ ID NO: 19 - DNA, 20 bp, Artificial sequence
  Single strand DNA oligonucleotide
cctgactccatttttgagcc

SEQ ID NO: 20 - DNA, 22 bp, Artificial sequence
  Single strand DNA oligonucleotide
cccaaatataacacgcttcact

SEQ ID NO: 21 - DNA, 23 bp, Artificial sequence
  Single strand DNA oligonucleotide
gaaatgaattattttctggcgac

SEQ ID NO: 22 - DNA, 20 bp, Artificial sequence
  Single strand DNA oligonucleotide
agctctttctcgattccgtg

SEQ ID NO: 23 - DNA, 20 bp, Artificial sequence
  Single strand DNA oligonucleotide
gggtagacacaagctgagcc

SEQ ID NO: 24 - DNA, 20 bp, Artificial sequence
  Single strand DNA oligonucleotide
caactatcaggctccaccac

SEQ ID NO: 25 - DNA, 20 bp, Artificial sequence
  Single strand DNA oligonucleotide
ctcagacggttttcagggtt

SEQ ID NO: 26 - DNA, 20 bp, Artificial sequence
  Single strand DNA oligonucleotide
aggctatgggatgatgcaag

SEQ ID NO: 27 - DNA, 20 bp, Artificial sequence
  Single strand DNA oligonucleotide
gtaggtaggcgatccgttca

SEQ ID NO: 28 - DNA, 20 bp, Artificial sequence
  Single strand DNA oligonucleotide
cgagaccaacgtgaagggaa

SEQ ID NO: 29 - DNA, 20 bp, Artificial sequence
  Single strand DNA oligonucleotide
cagacccggacaacgtcttt

SEQ ID NO: 30 - DNA, 20 bp, Artificial sequence
  Single strand DNA oligonucleotide
ctctggggctcacctacaag

SEQ ID NO: 31 - DNA, 20 bp, Artificial sequence
  Single strand DNA oligonucleotide
agccctggtgaaatgtaggg

SEQ ID NO: 32 - DNA, 20 bp, Artificial sequence
  Single strand DNA oligonucleotide
agctctcatctcaaggcaca

SEQ ID NO: 33 - DNA, 20 bp, Artificial sequence
  Single strand DNA oligonucleotide
ggaaagatccactgcttcca

SEQ ID NO: 34 - DNA, 22 bp, Artificial sequence
  Single strand DNA oligonucleotide
agcacaggtggtgaacgaacca

SEQ ID NO: 35 - DNA, 23 bp, Artificial sequence
  Single strand DNA oligonucleotide
tccaggcctcttgatgctaccga

SEQ ID NO: 36 - DNA, 24 bp, Artificial sequence
  Single strand DNA oligonucleotide
caccgccaaataaggcacgttatc

SEQ ID NO: 37 - DNA, 24 bp, Artificial sequence
  Single strand DNA oligonucleotide
aaacgataacgtgccttatttggc

SEQ ID NO: 38 - DNA, 25 bp, Artificial sequence
  Single strand DNA oligonucleotide
caccgaccagataacgtgccttatt

SEQ ID NO: 39 - DNA, 24 bp, Artificial sequence
  Single strand DNA oligonucleotide
aaacaataaggcacgttatctggt

SEQ ID NO: 40 - DNA, 19 bp, Artificial sequence
  Single strand DNA oligonucleotide
ttgcagtggttaccgttcg

SEQ ID NO: 41 - DNA, 20 bp, Artificial sequence
  Single strand DNA oligonucleotide
tagtaggcatcttgtggggg

SEQ ID NO: 42 - DNA, 20 bp, Artificial sequence
  Single strand DNA oligonucleotide
atgaatggagctataggagg

SEQ ID NO: 43 - DNA, 61 bp, Artificial sequence
  Single strand DNA oligonucleotide
ccacgtctcctgcttgctttaacagagagaagttcgtggcatcgccatcttccagcaggcg

SEQ ID NO: 44 - DNA, 61 bp, Artificial sequence
  Single strand DNA oligonucleotide
tgttaaagcaagcaggagacgtggaagaaaaccccggtcctatgaagatggacaaaaagac

SEQ ID NO: 45 - DNA, 21 bp, Artificial sequence
  Single strand DNA oligonucleotide
ttacagcccggaccgacgatg

SEQ ID NO: 46 - DNA, 32 bp, Artificial sequence
  Single strand DNA oligonucleotide
atctgacccgggatgaatggagctataggagg

SEQ ID NO: 47 - DNA, 33 bp, Artificial sequence
  Single strand DNA oligonucleotide
gtagctgctagcttacagcccggaccgacgatg

SEQ ID NO: 48 - DNA, 31 bp, Artificial sequence
  Single strand DNA oligonucleotide
caggtccccgggatggatcctgatgatgttg

SEQ ID NO: 49 - DNA, 31 bp, Artificial sequence
  Single strand DNA oligonucleotide
gcatgtgctagcttagagctttaaatctctg

SEQ ID NO: 50 - DNA, 9289 bp, Artificial sequence
  pX330-GFP plasmid nucleic acid sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 51 - DNA, 9291 bp, Artificial sequence
  CRISPR1 nucleic acid sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccaaataaggcacgttatcgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 52 - DNA, 9292 bp, Artificial sequence
  CRISPR3 nucleic acid sequence
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaccagataacgtgccttattgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 53 - DNA, 6061 bp, Artificial sequence
  pJet-Has nucleic acid sequence
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatttttgaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcataccagataacgtgccttatttggccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaaccatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 54 - DNA, 6929 bp, Artificial sequence
  pCAGG-Neo-IRES-GFP nucleic acid sequence
tcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatctagcctccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggg

SEQ ID NO: 55 - DNA, 10461 bp, Artificial sequence
  Targeting Vector nucleic acid sequence
gcccctgcagccgaattatattatttttgccaaataatttttaacaaaagctctgaagtcttcttcatttaaattcttagatgatacttcatctggaaaattgtcccaattagtagcatcacgctgtgagtaagttctaaaccatttttttattgttgtattatctctaatcttactactcgatgagttttcggtattatctctatttttaacttggagcaggttccattcattgtttttttcatcatagtgaataaaatcaactgctttaacacttgtgcctgaacaccatatccatccggcgtaatacgactcactatagggagagcggccgccagatcttccggatggctcgagtttttcagcaagatttttgaatgaagggcctgagggtgggcagtctgtctatcatgtacatctccatattctgggaggtcgtcagttgggctggcctcctggctaagatttttgcaccacaagagatgctgcatgtgtacaaatcactagcaaatagatttgtttcccatcaacttagccactgttaatgtaaattgttcttggatatgtgtctttggagggcaataaatgctctgaacagcacttgcacaataaagatacagcatgtgggaatgatctgtctcatgtgtcttactgatggtattggttctgtaagataaaatattgtgtctgggatgtgtttggctctactattaatggtgctctattgattgtgatttgtcatttgaaacctgaggatgcgactgtatagcagtctttcatgcatttttggaaaaaaacttaagctttttgaaagctgctgctacaactttttgtattgttataaagttttgtattgtttttttaattgtgaaattataaagatgccgtgcagggactgtttgaagcaaagtgcattgttttagaaacctacaactctagttcaagcactccatcagtatctgcttaatctttgtcatcctttgctatgagaaaatattaagcagtagtctaaaggtactatgaaactataacatagctgacattgtatttataactacgtcatgattttgatagaattgaggacttgaaaatgttaaactattcatgtagggcctcttaagatgcttaagttgtttagtaatgtaagtgtgcatttaattgagattttattgggcataatttgtccatcagtatgacactccttgtcagtgttgccttatacttgatgttgttaccggatctctgcaaggcagttattcttgaattaggctcattgaagtgtctgccagtataaatatatagcaactgttctttgtgttaaaattgagaagctaaccagtttttagtgcttctgactgttggaattctttaagcagatgccataagaaaattgtatttgtgatcaccacttctccagagtggttttaacaccaagggcattagagaaagaaaggcaggcgtgtagagaatagtggacagacaaaagctgtgagttacgttatgtttttcagctgaaaagctgtgtttggtaaaagcatatgaaatcactcaacttggaagcattctcttagttctctgatagttctgagcagcagaactcttcacctaagaggttacttcaactggaagactacctagtgcttctgatggcaactatatttaagatgagaataagaggtgtttccagtgtggtagcctcacatctgttgcagtggttaccgttcgtcctcctccgagggacacagcttggccattcactgtggtgacaccaatatgatgatcagcaaatggtgtttattcactactaaacacagcttatatacatttttacctacaaaatcgtgctgtcatgtcccactctgattggttcacacatttccccgaaaagtgccacctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatctagcctccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgccgtttccacattcttttctcatccttcttctcctgttttctctgcatcaaggtcagcacgatagcactgtctctctatgcttagggagaggcctgtcctgtacatcccgtgcccccacaagatgcctactacaacaacatcttctgcatgtcctgcatagcagtgttgggagaatgtgcactacttccactcttctgatttctattttatgtgtttgctttataccagtgttgccatttgggaattaatacatggttgatcaaatcaattgcatcacagctgtatcctgtatcagaggaacattatcaaagcttttgttgctgtatttggtatctgacctgcagataaacatgttttaggaaggttttgcaaaagtagctgtgaaatgagctggtgttgtgatttaacctgacaggcagctaaacagtataccacagagctattcacctactttccctcagtgggaaaagggaagagaactgaggggggggggaataaataagtaaataacaaaataaaactcatggattaagaaaaagactttgtactggaatggatgagaagaataatagtaatgataataatatgtcactctgaaagtaatgcctcttatttctgtggagactacaaacatacaaagagcacaacattccatagagcaaattctcagttacagaatgctattttttttttcaacacagtcaaaatcattaatttttttttgcctgcaatggacaagagctttgaagctgttctcgtaaaaatctgtactagcagaagtgacctgcaatcactactgctgaaatgcacaacccaccacatcattgtgctcacattcactgtttggtttctgtaaatgtacaggaattgtctgaaattagatatgattttttttttctccatgaaggaattcaattacacacctttgcctcatgcacttctttgtcatttttgtcagactgcttctctcctgcaatttgtctcatggcaacaaaatataatggagttctgctgggaacttccctactgccataccactatcatctgcctctgacattttggacaaatgtaataaaataggaggtattactttcagagcagaccttgtatgtatttacaaaacaagtggtacacaaaaaaaattgttcatcccaccaaccaatgcccatcctgtccctgaatagtagctgtcccccacagccttgaccagtttaggtcaacagttctgcttctgtcccctcccagctccttgtaacccctcagccccccttgctggcaggacagtatgagaagctgaaaaactagaatgtcctagttctttgcagtgctgctaatcaacaaccaaaacagtggtgtgttaccaatattgttgatatcacagcatcataccattatgaaggaagtaacccagccaaaatcaggtcagcttgctaacaagagaactgtgcataagtttaagatgtgtgtgttcctcagtaccttaaaaaataagtagtaacgttcaaatgagtagaagagtagaactgagcttaaaacatctgtcagacaacagtgaaccaaccatctttctagaagatctcctacaatattctcagctgccatggaaaatcgatgttcttcttttattctctcaagattttcaggctgtatattaaaacttatattaagaactatgctaaccacctcatcaggaaccgttgtaggtggcgtgggttttcttggcaatcgactctcatgaaaactacgagctaaatattcaatatgttcctcttgaccaactttattctgcattttttttgaacgaggtttagagcaagcttcaggaaactgagacaggaattttattaaaaatttaaattttgaagaaagttcagggttaatagcatccattttttgctttgcaagttcctcagcattcttaacaaaagacgtctcttttgacatgtttaaagtttaaacctcctgtgtgaaattattatccgctcataattccacacattatacgagccggaagcataaagtgtaaagcctggggtgcctaatgagtgagctaactcacattaattgcgttgcgctcactgccaattgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcc

SEQ ID NO: 56 - DNA, 7679 bp, Artificial sequence
  pmCherry-Cry2-CreN nucleic acid sequence
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggataggatcaagcttatcgataccgtcgacctcgagctcgccaccatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttcgttacaacgggtcaaagagagtgaaacctgaggaagaagaagagagagacatgaagaaatctaggggattcgatgaaagggagttgttttcgactgctgaatcttcttcttcttcgagtgtgtttttcgtttcgcagtcttgctcgttggcatcagaagggaagaatctggaaggtattcaagattcatctgatcagattactacaagtttgggaaaaaatggttgcaaaggtggcggtggctctggaggtggtgggtccggaggaggcggccgcacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgtaacccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggac

SEQ ID NO: 57 - DNA, 6655 bp, Artificial sequence
  pmCherry-CIBN-CreC nucleic acid sequence
  misc_feature (1717)..(1717): n is a, c, g, or t
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacntaagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggataggatcaagcttatcgataccgtcgacctcgagctcgccaccatgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaagaaacggaagtttgatacagagactaaggattgtaatgagaagaagaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatggtggcggtggctctggaggtggtgggtccggaggaggcggccgccgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcattagcaccgcctacatacctcgctctgctaatcctgttaccagtggctgctgccagtggcgataagtcgtgtcttaccgggttgga

SEQ ID NO: 58 - DNA, 8968 bp, Artificial sequence
  pB-RAGE-GFP nucleic acid sequence
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctggctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccatcaagcttaggatccggaacccttaatataacttcgtataatgtatgctatacgaagttattaggtccctcgacctgcagcccaagcttacttaccatgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccctcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacatgagcaactacaataacaacaacggcggctacaacggtggccgtggcggtggcagcttctttagcaacaaccgtcgtggtggttacggcaacggtggtttcttcggtggaaacaacggtggcagcagatctaacggccgttctggtggtagatggatcgatggcaaacatgtcccagctccaagaaacgaaaaggccgagatcgccatatttggtgtccccgaggatccggaacccttaatataacttcgtataatgtatgctatacgaagttattaggtccctcgaagaggttcactagggctagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggagtggggtggggcaggacagcaagggggaggattgggaagacaatagcaggcatgctggggatgcggtgggctctatggcttctgaggcggaaagaaccagctggggcttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 59 - DNA, 6188 bp, Artificial sequence
  pCAGG-IRES-GFP nucleic acid sequence
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcacctgcacatgggacgtcgacctgaggtaattataacccgggccctatatatggatcggctagccgatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattg

SEQ ID NO: 60 - DNA, 9644 bp, Artificial sequence
  pCAGG-Optogenes nucleic acid sequence
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcacctgcacatgggacgtcgacctgaggtaattataaccccccgggatgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaagaaacggaagtttgatacagagactaaggattgtaatgagaagaagaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatggtggcggtggctctggaggtggtgggtccggaggaggcggccgccgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatgccacgaacttctctctgttaaagcaagcaggagacgtggaagaaaaccccggtcctatgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttcgttacaacgggtcaaagagagtgaaacctgaggaagaagaagagagagacatgaagaaatctaggggattcgatgaaagggagttgttttcgactgctgaatcttcttcttcttcgagtgtgtttttcgtttcgcagtcttgctcgttggcatcagaagggaagaatctggaaggtattcaagattcatctgatcagattactacaagtttgggaaaaaatggttgcaaaggtggcggtggctctggaggtggtgggtccggaggaggcggccgcacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgtaagctagcctccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattg

SEQ ID NO: 61 - DNA, 9166 bp, Artificial sequence
  pB-RAGE-mCherry nucleic acid sequence
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctggctcgcgcgacttggtttgccattctttagcgcgcgtcgcgtcacacagcttggccacaatgtggtttttgtcaaacgaagattctatgacgtgtttaaagtttaggtcgagtaaagcgcaaatcttttttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtgactaatatataataaaatgggtagttctttagacgatgagcatatcctctctgctcttctgcaaagcgatgacgagcttgttggctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattccatcaagcttaggatccggaacccttaatataacttcgtataatgtatgctatacgaagttattaggtccctcgacctgcagcccaagcttacttaccatgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccctcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacatgagcaactacaataacaacaacggcggctacaacggtggccgtggcggtggcagcttctttagcaacaaccgtcgtggtggttacggcaacggtggtttcttcggtggaaacaacggtggcagcagatctaacggccgttctggtggtagatggatcgatggcaaacatgtcccagctccaagaaacgaaaaggccgagatcgccatatttggtgtccccgaggatccggaacccttaatataacttcgtataatgtatgctatacgaagttattaggtccctcgaagaggttcactagggctagcagttataggatctccgccaccatgctgtgctgcatcagaagaactaaaccggttgagaagaatgaagaggccgatcaggagctgcagtcgacggtaccgcgggcccgggatccaccggtagcatccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggcagtggagagggcagaggaagtctgctaacatgcggtgacgtcgaggagaatcctggcccaactagtgtttaaacgcggccgcggcaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggagtggggtggggcaggacagcaagggggaggattgggaagacaatagcaggcatgctggggatgcggtgggctctatggcttctgaggcggaaagaaccagctggggcttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 62 - DNA, 4526 bp, Artificial sequence
  pSK BS-PGK-DTA nucleic acid sequence
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctccaccgcggtggcggccgctctagaactagtggatccccaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcaggtcctcgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatatcaagcttatcgataccgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcag

SEQ ID NO: 63 - DNA, 4929 bp, Artificial sequence
  pGK-IRES-GFP nucleic acid sequence
tcgacaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgccccgggccctatatatggatcggctagccgatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggg

SEQ ID NO: 64 - DNA, 5568 bp, Artificial sequence
  pGK-DTA-IRES-GFP nucleic acid sequence
tcgacaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgccccgggatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaagctagcctccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggg

SEQ ID NO: 65 - DNA, 9 bp, Artificial sequence
  CRISPR1 cleavage site nucleic acid sequence
agataacgt

SEQ ID NO: 66 - DNA, 20 bp, Artificial sequence
  sgRNA nucleic acid sequence
gccaaataaggcacgttatc

SEQ ID NO: 67 - DNA, 20 bp, Artificial sequence
  sgRNA nucleic acid sequence
aatgtggaaacggccaaata

SEQ ID NO: 68 - DNA, 20 bp, Artificial sequence
  sgRNA nucleic acid sequence
accagataacgtgccttatt

SEQ ID NO: 69 - DNA, 20 bp, Artificial sequence
  sgRNA nucleic acid sequence
acatgacagcacgattttgt

SEQ ID NO: 70 - DNA, 20 bp, Artificial sequence
  sgRNA nucleic acid sequence
ctggtatgaaccaatcagag

SEQ ID NO: 71 - DNA, 20 bp, Artificial sequence
  sgRNA nucleic acid sequence
tggtatgaaccaatcagagt

SEQ ID NO: 72 - DNA, 20 bp, Artificial sequence
  sgRNA nucleic acid sequence
gaccttgatgcagagaaaac

SEQ ID NO: 73 - DNA, 20 bp, Artificial sequence
  sgRNA nucleic acid sequence
ctcctgttttctctgcatca

SEQ ID NO: 74 - DNA, 20 bp, Artificial sequence
  sgRNA nucleic acid sequence
gcagagaaaacaggagaaga

SEQ ID NO: 75 - DNA, 20 bp, Artificial sequence
  sgRNA nucleic acid sequence
agaaggatgagaaaagaatg

SEQ ID NO: 76 - DNA, 20 bp, Artificial sequence
  sgRNA nucleic acid sequence
ctgtcatgtcccactctgat

SEQ ID NO: 77 - DNA, 20 bp, Artificial sequence
  sgRNA nucleic acid sequence
atgagaaaagaatgtggaaa

SEQ ID NO: 78 - DNA, 23 bp, Artificial sequence
  off-targets nucleic acid sequence for guide #1
ccaacagaaggcacgttatccag

SEQ ID NO: 79 - DNA, 23 bp, Artificial sequence
  off-targets nucleic acid sequence for guide #1
tcaaaataaagtacgttatctag

SEQ ID NO: 80 - DNA, 23 bp, Artificial sequence
  off-targets nucleic acid sequence for guide #1
ggcatataaagcacgttatacag

SEQ ID NO: 81 - DNA, 23 bp, Artificial sequence
  off-targets nucleic acid sequence for guide #1
gcataataatgtacgttatctgg

SEQ ID NO: 82 - DNA, 23 bp, Artificial sequence
  off-targets nucleic acid sequence for guide #1
actaaatcaggcacgtgatctgg

SEQ ID NO: 83 - DNA, 23 bp, Artificial sequence
  off-targets nucleic acid sequence for guide #1
gctaaattaagctcgttatcggg

SEQ ID NO: 84 - DNA, 23 bp, Artificial sequence
  off-targets nucleic acid sequence for guide #1
gtcaaatgaggcatgttatcagg

SEQ ID NO: 85 - DNA, 23 bp, Artificial sequence
  off-targets nucleic acid sequence for guide #1
ttcaaataagccacgttattcag

SEQ ID NO: 86 - DNA, 23 bp, Artificial sequence
  off-targets nucleic acid sequence for guide #1
gtcaaacaaggcatgttatcagg

SEQ ID NO: 87 - DNA, 23 bp, Artificial sequence
  off-targets nucleic acid sequence for guide #1
ccctaataaagcacgttttcagg